SEQUENCE LISTING (130 sequences)

SEQ ID NO: 1 - DNA, 60 bp, Artificial Sequence
  Synthetic oligonucleotide
ggatattcctttctactctttgacatcatctatcatgttttcatggcaaaccctaacatc

SEQ ID NO: 2 - DNA, 59 bp, Artificial Sequence
  Synthetic oligonucleotide
ggatattcctttctactctttgacatcatctatcacaaagtactggcctttgctgctta

SEQ ID NO: 3 - DNA, 63 bp, Artificial Sequence
  Synthetic oligonucleotide
ggatattcctttctactctttgacatcatctatcacatgagagggctctaaataatctccttc

SEQ ID NO: 4 - DNA, 63 bp, Artificial Sequence
  Synthetic oligonucleotide
ggatattcctttctactctttgacatcatctatcaccaacaggtattgattcttgaggactta

SEQ ID NO: 5 - DNA, 66 bp, Artificial Sequence
  Synthetic oligonucleotide
ggatattcctttctactctttgacatcatctatcacagtgttttctttatttttatgtccaggtca

SEQ ID NO: 6 - DNA, 67 bp, Artificial Sequence
  Synthetic oligonucleotide
ggatattcctttctactctttgacatcatctatcaatttttcatattttttttccagagggaaacag

SEQ ID NO: 7 - DNA, 59 bp, Artificial Sequence
  Synthetic oligonucleotide
ggatattcctttctactctttgacatcatctatcatctgcaccatgttcctccaactat

SEQ ID NO: 8 - DNA, 59 bp, Artificial Sequence
  Synthetic oligonucleotide
ggatattcctttctactctttgacatcatctatcatgtgctactctgaccaagacagaa

SEQ ID NO: 9 - DNA, 57 bp, Artificial Sequence
  Synthetic oligonucleotide
ggatattcctttctactctttgacatcatctatcactgtgcagacataatggcagga

SEQ ID NO: 10 - DNA, 53 bp, Artificial Sequence
  Synthetic oligonucleotide
ggatattcctttctactctttgacatcatctatcacggaacggtcgaggcgaa

SEQ ID NO: 11 - DNA, 54 bp, Artificial Sequence
  Synthetic oligonucleotide
cctatggtagttaaatgtacattggatattcctttctactctttgacatcatct

SEQ ID NO: 12 - DNA, 58 bp, Artificial Sequence
  Synthetic oligonucleotide
agacgtgtgctcttccgatctatcahhhhhhhhhhhhhhhgtgcaagctggaggcact

SEQ ID NO: 13 - DNA, 69 bp, Artificial Sequence
  Synthetic oligonucleotide
agacgtgtgctcttccgatctatcahhhhhhhhhhhhhhhacaggaaaagaaactaaaattgtaccctt

SEQ ID NO: 14 - DNA, 63 bp, Artificial Sequence
  Synthetic oligonucleotide
agacgtgtgctcttccgatctatcahhhhhhhhhhhhhhhgaagccagatctcaaagtgtcct

SEQ ID NO: 15 - DNA, 67 bp, Artificial Sequence
  Synthetic oligonucleotide
agacgtgtgctcttccgatctatcahhhhhhhhhhhhhhhgttattgggaatgctatgaaagagaca

SEQ ID NO: 16 - DNA, 59 bp, Artificial Sequence
  Synthetic oligonucleotide
agacgtgtgctcttccgatctatcahhhhhhhhhhhhhhhggctgcgatgagacaggaa

SEQ ID NO: 17 - DNA, 71 bp, Artificial Sequence
  Synthetic oligonucleotide
agacgtgtgctcttccgatctatcahhhhhhhhhhhhhhhtgaaagatagcaatagatacataaaacacca

SEQ ID NO: 18 - DNA, 64 bp, Artificial Sequence
  Synthetic oligonucleotide
agacgtgtgctcttccgatctatcahhhhhhhhhhhhhhhcaatttccaagacagaagcactcc

SEQ ID NO: 19 - DNA, 65 bp, Artificial Sequence
  Synthetic oligonucleotide
agacgtgtgctcttccgatctatcahhhhhhhhhhhhhhhgggggaaaatggtttcttaggatga

SEQ ID NO: 20 - DNA, 56 bp, Artificial Sequence
  Synthetic oligonucleotide
agacgtgtgctcttccgatctatcahhhhhhhhhhhhhhhagcatgccgcccttgg

SEQ ID NO: 21 - DNA, 67 bp, Artificial Sequence
  Synthetic oligonucleotide
agacgtgtgctcttccgatctatcahhhhhhhhhhhhhhhtcacaggtcaaaattatgagttcttcg

SEQ ID NO: 22 - DNA, 32 bp, Artificial Sequence
  Synthetic oligonucleotide
gactggagttcagacgtgtgctcttccgatct

SEQ ID NO: 23 - DNA, 45 bp, Artificial Sequence
  Synthetic oligonucleotide
acacgacgctcttccgatctacttctgccaacattcaaattcagg

SEQ ID NO: 24 - DNA, 43 bp, Artificial Sequence
  Synthetic oligonucleotide
acacgacgctcttccgatctggatgggactccaatgcaaaact

SEQ ID NO: 25 - DNA, 48 bp, Artificial Sequence
  Synthetic oligonucleotide
acacgacgctcttccgatctcatcttgctcttcatagatagcttcaga

SEQ ID NO: 26 - DNA, 47 bp, Artificial Sequence
  Synthetic oligonucleotide
acacgacgctcttccgatctcctgaatgtcagttttgttagagcaac

SEQ ID NO: 27 - DNA, 44 bp, Artificial Sequence
  Synthetic oligonucleotide
acacgacgctcttccgatctctccttggaatcaccaacaaacat

SEQ ID NO: 28 - DNA, 43 bp, Artificial Sequence
  Synthetic oligonucleotide
acacgacgctcttccgatctttcctgcttccagacatgaatca

SEQ ID NO: 29 - DNA, 45 bp, Artificial Sequence
  Synthetic oligonucleotide
acacgacgctcttccgatctctctctagagtgcagattggtagaa

SEQ ID NO: 30 - DNA, 45 bp, Artificial Sequence
  Synthetic oligonucleotide
acacgacgctcttccgatctacatgtccaaagagagaagtcgtag

SEQ ID NO: 31 - DNA, 40 bp, Artificial Sequence
  Synthetic oligonucleotide
acacgacgctcttccgatctgccccaaaggttaccccatg

SEQ ID NO: 32 - DNA, 41 bp, Artificial Sequence
  Synthetic oligonucleotide
acacgacgctcttccgatctgtagccgcttctctgtgagtt

SEQ ID NO: 33 - DNA, 25 bp, Artificial Sequence
  Synthetic oligonucleotide
acttctgccaacattcaaattcagg

SEQ ID NO: 34 - DNA, 23 bp, Artificial Sequence
  Synthetic oligonucleotide
ggatgggactccaatgcaaaact

SEQ ID NO: 35 - DNA, 28 bp, Artificial Sequence
  Synthetic oligonucleotide
catcttgctcttcatagatagcttcaga

SEQ ID NO: 36 - DNA, 27 bp, Artificial Sequence
  Synthetic oligonucleotide
cctgaatgtcagttttgttagagcaac

SEQ ID NO: 37 - DNA, 24 bp, Artificial Sequence
  Synthetic oligonucleotide
ctccttggaatcaccaacaaacat

SEQ ID NO: 38 - DNA, 23 bp, Artificial Sequence
  Synthetic oligonucleotide
ttcctgcttccagacatgaatca

SEQ ID NO: 39 - DNA, 25 bp, Artificial Sequence
  Synthetic oligonucleotide
ctctctagagtgcagattggtagaa

SEQ ID NO: 40 - DNA, 25 bp, Artificial Sequence
  Synthetic oligonucleotide
acatgtccaaagagagaagtcgtag

SEQ ID NO: 41 - DNA, 20 bp, Artificial Sequence
  Synthetic oligonucleotide
gccccaaaggttaccccatg

SEQ ID NO: 42 - DNA, 21 bp, Artificial Sequence
  Synthetic oligonucleotide
gtagccgcttctctgtgagtt

SEQ ID NO: 43 - DNA, 34 bp, Artificial Sequence
  Synthetic oligonucleotide
tcaaattcaggtaccttagagggacagctaaact

SEQ ID NO: 44 - DNA, 35 bp, Artificial Sequence
  Synthetic oligonucleotide
aatgcaaaactcaatgtatcagtgtgaggatgtat

SEQ ID NO: 45 - DNA, 33 bp, Artificial Sequence
  Synthetic oligonucleotide
tagcttcagaaacattccagtgtatgtgcagga

SEQ ID NO: 46 - DNA, 34 bp, Artificial Sequence
  Synthetic oligonucleotide
gttagagcaactttccttgattcccagagtagct

SEQ ID NO: 47 - DNA, 30 bp, Artificial Sequence
  Synthetic oligonucleotide
caacaaacatgccttctccttctcctgaaa

SEQ ID NO: 48 - DNA, 33 bp, Artificial Sequence
  Synthetic oligonucleotide
acatgaatcatgtcactattcaatgggatgctt

SEQ ID NO: 49 - DNA, 33 bp, Artificial Sequence
  Synthetic oligonucleotide
gattggtagaagacactgattgcatcttcaagt

SEQ ID NO: 50 - DNA, 31 bp, Artificial Sequence
  Synthetic oligonucleotide
gaagtcgtagctattcggcaaaggaaatgtt

SEQ ID NO: 51 - DNA, 31 bp, Artificial Sequence
  Synthetic oligonucleotide
taccccatgtgtatcaaatggtcagcaagtt

SEQ ID NO: 52 - DNA, 26 bp, Artificial Sequence
  Synthetic oligonucleotide
ctgtgagttgggagcaaaggagcaat

SEQ ID NO: 53 - DNA, 53 bp, Artificial Sequence
  Synthetic oligonucleotide
ggatattcctttctactctttgacatcatctatcaaagcgccttgaggccttt

SEQ ID NO: 54 - DNA, 54 bp, Artificial Sequence
  Synthetic oligonucleotide
ggatattcctttctactctttgacatcatctatcaaagggagctgcaggttctg

SEQ ID NO: 55 - DNA, 50 bp, Artificial Sequence
  Synthetic oligonucleotide
ggatattcctttctactctttgacatcatctatcactgcccgctgacccc

SEQ ID NO: 56 - DNA, 54 bp, Artificial Sequence
  Synthetic oligonucleotide
ggatattcctttctactctttgacatcatctatcattgagttcgccgaccttgg

SEQ ID NO: 57 - DNA, 51 bp, Artificial Sequence
  Synthetic oligonucleotide
ggatattcctttctactctttgacatcatctatcacgcgagctgcaggtcc

SEQ ID NO: 58 - DNA, 58 bp, Artificial Sequence
  Synthetic oligonucleotide
ggatattcctttctactctttgacatcatctatcatccaacatcctcgtgaactctag

SEQ ID NO: 59 - DNA, 55 bp, Artificial Sequence
  Synthetic oligonucleotide
ggatattcctttctactctttgacatcatctatcaggcccaacaccttcatcatc

SEQ ID NO: 60 - DNA, 60 bp, Artificial Sequence
  Synthetic oligonucleotide
ggatattcctttctactctttgacatcatctatcatgtcttcaaaaggaagtatcttggc

SEQ ID NO: 61 - DNA, 55 bp, Artificial Sequence
  Synthetic oligonucleotide
ggatattcctttctactctttgacatcatctatcaattgtcagtgcgcttttccc

SEQ ID NO: 62 - DNA, 60 bp, Artificial Sequence
  Synthetic oligonucleotide
ggatattcctttctactctttgacatcatctatcactgtttgttggacatactggataca

SEQ ID NO: 63 - DNA, 56 bp, Artificial Sequence
  Synthetic oligonucleotide
ggatattcctttctactctttgacatcatctatcagtatcgtcaaggcactcttgc

SEQ ID NO: 64 - DNA, 59 bp, Artificial Sequence
  Synthetic oligonucleotide
ggatattcctttctactctttgacatcatctatcactgtctcttggatattctcgacac

SEQ ID NO: 65 - DNA, 60 bp, Artificial Sequence
  Synthetic oligonucleotide
ggatattcctttctactctttgacatcatctatcatcaaagcaatttctacacgagatcc

SEQ ID NO: 66 - DNA, 61 bp, Artificial Sequence
  Synthetic oligonucleotide
ggatattcctttctactctttgacatcatctatcaaggctttggagtatttcatgaaacaa

SEQ ID NO: 67 - DNA, 55 bp, Artificial Sequence
  Synthetic oligonucleotide
ggatattcctttctactctttgacatcatctatcaactgatgggacccactccat

SEQ ID NO: 68 - DNA, 54 bp, Artificial Sequence; identical to SEQ ID NO: 11
  Synthetic oligonucleotide

SEQ ID NO: 69 - DNA, 59 bp, Artificial Sequence
  Synthetic oligonucleotide
agacgtgtgctcttccgatctatcahhhhhhhhhhhhhhhggcttgtgggagaccttga

SEQ ID NO: 70 - DNA, 60 bp, Artificial Sequence
  Synthetic oligonucleotide
agacgtgtgctcttccgatctatcahhhhhhhhhhhhhhhagccacccaactcttaaggc

SEQ ID NO: 71 - DNA, 67 bp, Artificial Sequence
  Synthetic oligonucleotide
agacgtgtgctcttccgatctatcahhhhhhhhhhhhhhhagttccctccttttctattttctcttc

SEQ ID NO: 72 - DNA, 62 bp, Artificial Sequence
  Synthetic oligonucleotide
agacgtgtgctcttccgatctatcahhhhhhhhhhhhhhhagtctccctaggtagctaaccc

SEQ ID NO: 73 - DNA, 59 bp, Artificial Sequence
  Synthetic oligonucleotide
agacgtgtgctcttccgatctatcahhhhhhhhhhhhhhhggacgcactcaccatgtgt

SEQ ID NO: 74 - DNA, 60 bp, Artificial Sequence
  Synthetic oligonucleotide
agacgtgtgctcttccgatctatcahhhhhhhhhhhhhhhgggactcacagccatgtagg

SEQ ID NO: 75 - DNA, 57 bp, Artificial Sequence
  Synthetic oligonucleotide
agacgtgtgctcttccgatctatcahhhhhhhhhhhhhhhccatccccgtgtccctc

SEQ ID NO: 76 - DNA, 67 bp, Artificial Sequence
  Synthetic oligonucleotide
agacgtgtgctcttccgatctatcahhhhhhhhhhhhhhhccagtgttgtaggacatatattgtacc

SEQ ID NO: 77 - DNA, 66 bp, Artificial Sequence
  Synthetic oligonucleotide
agacgtgtgctcttccgatctatcahhhhhhhhhhhhhhhgctttaaagtactgtagatgtggctc

SEQ ID NO: 78 - DNA, 64 bp, Artificial Sequence
  Synthetic oligonucleotide
agacgtgtgctcttccgatctatcahhhhhhhhhhhhhhhggttaatatccgcaaatgacttgc

SEQ ID NO: 79 - DNA, 70 bp, Artificial Sequence
  Synthetic oligonucleotide
agacgtgtgctcttccgatctatcahhhhhhhhhhhhhhhtgtattaaccttatgtgtgacatgttctaa

SEQ ID NO: 80 - DNA, 72 bp, Artificial Sequence
  Synthetic oligonucleotide
agacgtgtgctcttccgatctatcahhhhhhhhhhhhhhhttatattcaatttaaacccacctataatggtg

SEQ ID NO: 81 - DNA, 66 bp, Artificial Sequence
  Synthetic oligonucleotide
agacgtgtgctcttccgatctatcahhhhhhhhhhhhhhhggtatggtaaaaacatgctgagatca

SEQ ID NO: 82 - DNA, 60 bp, Artificial Sequence
  Synthetic oligonucleotide
agacgtgtgctcttccgatctatcahhhhhhhhhhhhhhhcagtgcagtgtggaatccag

SEQ ID NO: 83 - DNA, 73 bp, Artificial Sequence
  Synthetic oligonucleotide
agacgtgtgctcttccgatctatcahhhhhhhhhhhhhhhttacttactacacctcagatatatttcttcatg

SEQ ID NO: 84 - DNA, 32 bp, Artificial Sequence; identical to SEQ ID NO: 22
  Synthetic oligonucleotide

SEQ ID NO: 85 - DNA, 41 bp, Artificial Sequence
  Synthetic oligonucleotide
acacgacgctcttccgatctccttgaggcctttcttaccca

SEQ ID NO: 86 - DNA, 39 bp, Artificial Sequence
  Synthetic oligonucleotide
acacgacgctcttccgatctagctgcaggttctgcatga

SEQ ID NO: 87 - DNA, 39 bp, Artificial Sequence
  Synthetic oligonucleotide
acacgacgctcttccgatctcgctgaccccaaagtcaca

SEQ ID NO: 88 - DNA, 36 bp, Artificial Sequence
  Synthetic oligonucleotide
acacgacgctcttccgatctttcgccgaccttggct

SEQ ID NO: 89 - DNA, 37 bp, Artificial Sequence
  Synthetic oligonucleotide
acacgacgctcttccgatctgctgcaggtcctgcacg

SEQ ID NO: 90 - DNA, 41 bp, Artificial Sequence
  Synthetic oligonucleotide
acacgacgctcttccgatctcatcctcgtgaactctagagg

SEQ ID NO: 91 - DNA, 41 bp, Artificial Sequence
  Synthetic oligonucleotide
acacgacgctcttccgatctaacaccttcatcatccgctgc

SEQ ID NO: 92 - DNA, 44 bp, Artificial Sequence
  Synthetic oligonucleotide
acacgacgctcttccgatcttcaaaaggaagtatcttggcctcc

SEQ ID NO: 93 - DNA, 39 bp, Artificial Sequence
  Synthetic oligonucleotide
acacgacgctcttccgatctcagtgcgcttttcccaaca

SEQ ID NO: 94 - DNA, 43 bp, Artificial Sequence
  Synthetic oligonucleotide
acacgacgctcttccgatcttgttggacatactggatacagct

SEQ ID NO: 95 - DNA, 40 bp, Artificial Sequence
  Synthetic oligonucleotide
acacgacgctcttccgatctgtcaaggcactcttgcctac

SEQ ID NO: 96 - DNA, 43 bp, Artificial Sequence
  Synthetic oligonucleotide
acacgacgctcttccgatcttcttggatattctcgacacagca

SEQ ID NO: 97 - DNA, 44 bp, Artificial Sequence
  Synthetic oligonucleotide
acacgacgctcttccgatctgcaatttctacacgagatcctctc

SEQ ID NO: 98 - DNA, 50 bp, Artificial Sequence
  Synthetic oligonucleotide
acacgacgctcttccgatctttggagtatttcatgaaacaaatgaatgat

SEQ ID NO: 99 - DNA, 41 bp, Artificial Sequence
  Synthetic oligonucleotide
acacgacgctcttccgatcttgggacccactccatcgagat

SEQ ID NO: 100 - DNA, 21 bp, Artificial Sequence
  Synthetic oligonucleotide
ccttgaggcctttcttaccca

SEQ ID NO: 101 - DNA, 19 bp, Artificial Sequence
  Synthetic oligonucleotide
agctgcaggttctgcatga

SEQ ID NO: 102 - DNA, 19 bp, Artificial Sequence
  Synthetic oligonucleotide
cgctgaccccaaagtcaca

SEQ ID NO: 103 - DNA, 16 bp, Artificial Sequence
  Synthetic oligonucleotide
ttcgccgaccttggct

SEQ ID NO: 104 - DNA, 17 bp, Artificial Sequence
  Synthetic oligonucleotide
gctgcaggtcctgcacg

SEQ ID NO: 105 - DNA, 21 bp, Artificial Sequence
  Synthetic oligonucleotide
catcctcgtgaactctagagg

SEQ ID NO: 106 - DNA, 21 bp, Artificial Sequence
  Synthetic oligonucleotide
aacaccttcatcatccgctgc

SEQ ID NO: 107 - DNA, 24 bp, Artificial Sequence
  Synthetic oligonucleotide
tcaaaaggaagtatcttggcctcc

SEQ ID NO: 108 - DNA, 19 bp, Artificial Sequence
  Synthetic oligonucleotide
cagtgcgcttttcccaaca

SEQ ID NO: 109 - DNA, 23 bp, Artificial Sequence
  Synthetic oligonucleotide
tgttggacatactggatacagct

SEQ ID NO: 110 - DNA, 20 bp, Artificial Sequence
  Synthetic oligonucleotide
gtcaaggcactcttgcctac

SEQ ID NO: 111 - DNA, 23 bp, Artificial Sequence
  Synthetic oligonucleotide
tcttggatattctcgacacagca

SEQ ID NO: 112 - DNA, 24 bp, Artificial Sequence
  Synthetic oligonucleotide
gcaatttctacacgagatcctctc

SEQ ID NO: 113 - DNA, 30 bp, Artificial Sequence
  Synthetic oligonucleotide
ttggagtatttcatgaaacaaatgaatgat

SEQ ID NO: 114 - DNA, 21 bp, Artificial Sequence
  Synthetic oligonucleotide
tgggacccactccatcgagat

SEQ ID NO: 115 - DNA, 27 bp, Artificial Sequence
  Synthetic oligonucleotide
tcttacccagaagcagaaggtgggaaa

SEQ ID NO: 116 - DNA, 27 bp, Artificial Sequence
  Synthetic oligonucleotide
ctgcatgagtgcaactctccgtacagc

SEQ ID NO: 117 - DNA, 28 bp, Artificial Sequence
  Synthetic oligonucleotide
aaagtcacagagcttgatctccccacgc

SEQ ID NO: 118 - DNA, 27 bp, Artificial Sequence
  Synthetic oligonucleotide
ttggctttctgggtgagaaaggcttac

SEQ ID NO: 119 - DNA, 25 bp, Artificial Sequence
  Synthetic oligonucleotide
ctgcacgaatgcaactcgccgtata

SEQ ID NO: 120 - DNA, 28 bp, Artificial Sequence
  Synthetic oligonucleotide
tctagaggggagatcaagctgtgtgaaa

SEQ ID NO: 121 - DNA, 23 bp, Artificial Sequence
  Synthetic oligonucleotide
cgctgcctgcagtggaccactcc

SEQ ID NO: 122 - DNA, 36 bp, Artificial Sequence
  Synthetic oligonucleotide
gcctccagttttttatatattctcctacatgaggaa

SEQ ID NO: 123 - DNA, 24 bp, Artificial Sequence
  Synthetic oligonucleotide
cccaacaccacctgctccaaccct

SEQ ID NO: 124 - DNA, 30 bp, Artificial Sequence
  Synthetic oligonucleotide
gatacagctggacaagaagagtacagtgac

SEQ ID NO: 125 - DNA, 22 bp, Artificial Sequence
  Synthetic oligonucleotide
tgcctacgccaccagctccatt

SEQ ID NO: 126 - DNA, 28 bp, Artificial Sequence
  Synthetic oligonucleotide
cacagcaggtcaagaggagtacagtgac

SEQ ID NO: 127 - DNA, 29 bp, Artificial Sequence
  Synthetic oligonucleotide
atcctctctctgaaatcactgagcaggac

SEQ ID NO: 128 - DNA, 29 bp, Artificial Sequence
  Synthetic oligonucleotide
caaatgaatgatgcacatcatggtggcgt

SEQ ID NO: 129 - DNA, 33 bp, Artificial Sequence
  Synthetic oligonucleotide
ccatcgagatttcactgtagctagaccaaaaaa

SEQ ID NO: 130 - DNA, 34 bp, Artificial Sequence
  Synthetic oligonucleotide
ctgagatcagccaaagtcagttattttttctgac

(19) United States
(12) Patent Application Publication
(10) Pub. No.: US 2023/0220456 A1
(43) Pub. Date: Jul. 13, 2023

(54) QUANTITATIVE BLOCKER DISPLACEMENT AMPLIFICATION (QBDA) SEQUENCING FOR CALIBRATION-FREE AND MULTIPLEXED VARIANT ALLELE FREQUENCY QUANTITATION

(71) Applicant: William Marsh Rice University, Houston, TX (US)

(72) Inventors: David ZHANG, Houston, TX (US); Ruojia WU, Houston, TX (US); Peng DAI, Houston, TX (US); Xi CHEN, Houston, TX (US)

(73) Assignee: William Marsh Rice University, Houston, TX (US)

(21) Appl. No.: 17/997,696

(22) PCT Filed: Apr. 30, 2021
(86) PCT No.: PCT/US21/30249

§ 371 Date: Nov. 1, 2022

Related U.S. Application Data
(60) Provisional application No. 63/018,922, filed on May 1, 2020.

(51) Int. Cl. C12Q 1/6851; C12Q 1/686; C12Q 1/6869; C12Q 1/6876; C12Q 1/6858

(52) U.S. Cl. CPC C12Q 1/6851; C12Q 1/686; C12Q 1/6869; C12Q 1/6876; C12Q 1/6858; C12Q 2600/16

(57) ABSTRACT

Provided herein are methods for labeling input DNA with oligonucleotide barcode sequences, and selective PCR amplification of DNA sequence variants across the targeted regions for variant quantitation.

### REFERENCE TO RELATED APPLICATIONS

The present application claims the priority benefit of U.S. provisional application No. 63/018,922, filed May 1, 2020, the entire contents of which is incorporated herein by reference.

### STATEMENT REGARDING FEDERALLY SPONSORED RESEARCH

This invention was made with government support under Grant No. R01CA203964 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Apr. 28, 2021, is named RICEP0075WO_ST25.txt and is 27.8 kilobytes in size.

### BACKGROUND

The development of this disclosure was funded in part by the Cancer Prevention and Research Institute of Texas (CPRIT) under Grant No. RP180147.

### 1. Field

The present invention relates generally to the field of molecular biology. More particularly, it concerns compositions and methods for rare mutation quantitation and profiling using low-depth next-generation sequencing.

### 2. Description of Related Art

DNA variants with low allele frequency, such as cancer mutations and pathogen drug resistance mutations, hold important clinical and biological information, but are difficult to profile in a multiplexed and inexpensive way. Blocker Displacement Amplification (BDA)^{1,2} and multiplexed BDA (mBDA)³ provide PCR- and strand displacement-based methods to enrich hundreds of potentially rare mutations (≤0.1% variant allele frequency) in a single reaction. However, laborious calibration curves are required for accurate variant allele frequency quantitation in BDA. PCR-based methods for multiplex and calibration-free quantitation with low detection limits are needed.

### SUMMARY

As such, provided herein are PCR-based methods that add a unique molecular identifier (UMI) to each original molecule and allow for quantitative blocker displacement amplification (QBDA) featuring multiplex and calibration-free quantitation with a low detection limit. Any method provided herein is specifically envisioned for multiplex use.

In one embodiment provided herein are methods for labeling and amplifying each strand of between 1 and 10,000 target genomic regions with an oligonucleotide barcode sequence by polymerase chain reaction (PCR), the method comprising: (a) introducing into a DNA sample comprising the between 1 and 10,000 target genomic regions, for each target genomic region: (i) a first oligonucleotide, comprising from 5′ to 3′ end, (A) a first region, (B) a second region with a length between 0 and 50 nucleotides, and (C) a third region targeting a first specific genomic region; and (ii) a second oligonucleotide, comprising from 5′ to 3′ end, (A) a fourth region, (B) a fifth region with a length between 0 and 50 nucleotides, (C) a sixth region comprising a unique molecular identifier (UMI) comprising at least four degenerate nucleotides, and (D) a seventh region targeting a second specific genomic region; (b) performing at least two cycles of PCR amplification to generate a first PCR amplification product; (c) introducing into the first PCR amplification product: (i) a third oligonucleotide comprising the first region; and (ii) a fourth oligonucleotide, comprising the fourth region; (d) performing at least two cycles of PCR amplification, to generate a second PCR amplification product; (e) introducing to the second PCR amplification product: (i) a fifth oligonucleotide (Blocker Displacement Amplification (BDA) forward primer) for each target genomic region, wherein the BDA forward primer comprises an eighth region targeting a specific genomic region, wherein the genomic region targeted by the eighth region is between 1 and 20 nucleotides closer to the seventh region compared to the genomic region targeted by the third region, (ii) a sixth oligonucleotide (BDA blocker) for each target genomic region, wherein 4 or more nucleotides at the 3′ end of the BDA forward primer sequence are also present at or near the 5′ end of the BDA blocker sequence; and wherein the BDA blocker contains a 3′ sequence or modification that prevents extension by a DNA polymerase, and wherein the concentration of the BDA blocker is at least 2 times that of the BDA forward primer, and (iii) a seventh oligonucleotide, comprising the fourth region; and (f) performing at least two cycles of PCR amplification to generate a third PCR amplification product.

In one embodiment, provided herein are methods for labeling and amplifying each strand of at least one target genomic region with an oligonucleotide barcode sequence by polymerase chain reaction (PCR), the method comprising: (a) adding a unique molecular identifier (UMI) to the at least one target genomic region; (b) amplifying the at least one target genomic region from step (a) using a universal forward primer and a universal reverse primer, to generate a first PCR amplification product, wherein the at least one target genomic region comprises the UMI; and (c) amplifying the first PCR amplification product using a Blocker Displacement Amplification (BDA) forward primer, a BDA blocker, and a universal reverse primer to generate a second PCR amplification product.

In one embodiment, provided herein are methods for labeling and amplifying each strand of at least one target genomic region with an oligonucleotide barcode sequence by polymerase chain reaction (PCR), the method comprising: (a) introducing into a DNA sample comprising the at least one target genomic region: (i) a first oligonucleotide, comprising a third region targeting a first specific genomic region; and (ii) a second oligonucleotide, comprising a region comprising a unique molecular identifier (UMI) comprising at least four degenerate nucleotides, and a region targeting a second specific genomic region; (b) performing at least two cycles of PCR amplification to generate a first PCR amplification product; (c) introducing to the first PCR amplification product: (i) a universal forward primer; and (ii) a universal reverse primer; (d) performing at least two cycles of PCR amplification, to generate a second PCR amplification product; (e) introducing to the second PCR amplification product: (i) a Blocker Displacement Amplification (BDA) forward primer targeting the specific genomic region, wherein the genomic region targeted by the eighth region is between 1 and 20 nucleotides closer to the seventh region compared to the genomic region targeted by the third region, (ii) a BDA blocker for the target genomic region, wherein 4 or more nucleotides at the 3′ end of the BDA forward primer sequence are also present at or near the 5′ end of the BDA blocker sequence; and wherein the BDA blocker contains a 3′ sequence or modification that prevents extension by a DNA polymerase, and (iii) a universal reverse primer; and (f) performing at least two cycles of PCR amplification to obtain a third PCR amplification product. In some embodiments, the methods further comprise purifying the second PCR amplification product is purified between step (d) and step (e). In some embodiments, the methods further comprise wherein the concentration of the BDA blocker is at least 2 times that of the BDA forward primer.

In one embodiment, provided herein are methods for labeling and amplifying each strand of between 1 and 10,000 target genomic regions with an oligonucleotide barcode sequence by polymerase chain reaction (PCR), the methods comprising: (a) obtaining a DNA sample comprising the target genomic regions; (b) introducing into the DNA sample, for each target genomic region, (i) a first oligonucleotide, comprising from 5′ to 3′ end, a first region, a second region with a length between 0 and 50 nucleotides, and a third region targeting a specific genomic region, and (ii) a second oligonucleotide, comprising from 5′ to 3′ end, a fourth region, a fifth region with a length between 0 and 50 nucleotides, a sixth region comprising a unique molecular identifier (UMI) comprising at least four degenerate nucleotides, and a seventh region targeting a specific genomic region; (c) performing two cycles of PCR amplification to generate a first PCR amplification product; (d) introducing into the first PCR amplification product, (i) a third oligonucleotide, comprising the first region, and (ii) a fourth oligonucleotide, comprising the fourth region; (e) performing at least two cycles of PCR amplification, wherein the annealing temperature is between 0.01° C. and 10° C. higher than an annealing temperature used in step (c) to generate a second PCR amplification product; (f) purifying the second PCR amplification product to remove single-stranded primers; (g) introducing into the purified second PCR amplification product obtained in step (f), (i) a fifth oligonucleotide (BDA forward primer) for each target genomic region, comprising an eighth region targeting specific genomic region, wherein the genomic region targeted by the eighth region is 1-20 bases closer to the seventh region compared to the genomic region targeted by the third region, (ii) a sixth oligonucleotide (BDA blocker) for each target genomic region, wherein 4 or more nucleotides at the 3′ end of the BDA forward primer sequence are also present at or near the 5′ end of the BDA blocker sequence; and the BDA blocker contains a 3′ sequence or modification that prevents extension by DNA polymerase, and wherein the concentration of the BDA blocker is at least 2× that of the BDA forward primer, and (iii) a seventh oligonucleotide, comprising the fourth region; and (h) performing at least two cycles of PCR amplification to generate a third PCR amplification product.

In some aspects, the first region in the first oligonucleotide in step (b) and the fourth region in the second oligonucleotide in step (b) generate binding sites for universal amplification performed in step (d). In some aspects, the fourth region in the second oligonucleotide comprises at least part of the next-generation sequencing (NGS) adapter sequence. In some aspects, the melting temperatures of the first and the fourth regions are between 0.01° C. and 10° C. higher than the melting temperatures of the third and the seventh regions. In some aspects, the degenerate nucleotides in the sixth region each independently are one of A, T, or C. In some aspects, the purifying in step (f) comprises SPRI purification, column purification, or enzymatic digestion. In some aspects, step (b) further comprises introducing into the DNA sample a blocker oligonucleotide that comprises from 5′ to 3′ end, sequence that targets a pseudogene or other undesired genomic region and 3′ sequence or modification that prevents extension by DNA polymerase.

In some aspects, the methods further comprise (i) introducing to the PCR amplification product obtained in step (h), (i) an eighth oligonucleotide, comprising from 5′ to 3′ end, a ninth region and an eighth region, wherein the ninth region comprises at least part of the next-generation sequencing (NGS) adapter sequence, and optionally (ii) a ninth oligonucleotide, comprising the fourth region; and (j) performing at least one cycle of PCR amplification.

In some aspects, the methods further comprise (i) adding NGS adapter sequences to the PCR amplification product obtained in step (h) by ligation reaction.

In some aspects, the methods further comprise adding NGS indices by PCR and purifying the PCR product. In some aspects, the purifying comprises SPRI purification, column purification, or enzymatic digestion.

In some aspects, the methods further comprise performing high-throughput DNA sequencing. In some aspects, the high-throughput DNA sequencing is next-generation sequencing.

In one embodiment, provided herein are methods for quantitating the variant allele frequency (VAF) of variant sequences in between 1 and 10,000 genomic regions, the methods comprising: (a) designing a panel of oligonucleotides and blockers for the target genomic regions; (b) labeling and amplifying each strand of the targeted genomic regions according to the method of any one of the present embodiments; and (c) determining the variant allele frequency (VAF) of variant sequences based on high-throughput sequencing data and the input amount of DNA sample.

In some aspects, step (a) comprises: (i) designing a primer set for each selected genomic region; each primer set containing the first, the second, the fifth, the sixth, and the eighth oligonucleotide are as described in any of the present embodiments; (ii) designing the third and the fourth oligonucleotide to be used for universal amplification of all selected genomic regions; and (iii) checking the specificity of the primer set in whole genome to ensure that the primers are not prone to nonspecific amplification of non-target regions.

In some aspects, step (c) comprises: (i) aligning NGS reads to the targeted amplicon regions, and grouping the NGS reads into region-specific subgroups by the regions to which they are aligned; (ii) at each locus, dividing the NGS reads by the UMI sequence, where all NGS reads carrying the UMI sequence are grouped as one UMI family; (iii) removing UMI families that are likely results of PCR or NGS errors; (iv) determine the genotype for each remaining UMI family; (v) counting the unique UMI number N (the total count of different UMI sequences at one locus) for each variant sequence at each targeted genetic region, which indicates the number of original strands; and (vi) calculating VAF for a variant sequence as VAF=Nᵥₐᵣ/(Nᵢₙₚᵤₜ*Yield), where Nᵥₐᵣ is unique UMI number for the variant sequence, Nᵢₙₚᵤₜ is strand number of DNA input for QBDA, and Yield is the overall conversion yield for QBDA reaction.

In some aspects, UMI families are considered to be likely results of PCR or NGS errors if the UMI sequence does not meet the UMI degenerate base design pattern or the UMI family has a UMI family size <Fₘᵢₙ, wherein Fₘᵢₙ is between 2 and 20. In some aspects, step (iv) comprises determining the genotype supported by at least 70% of the reads in the same UMI family. In some aspects, step (iv) comprises determining the genotype as wild type (WT), if WT reads is supported by more than P_{WT} reads in the UMI family, wherein P_{WT} is 0.01%-50%. In some aspects, step (v) further comprises removing UMI sequences that differs by only 1 or 2 bases from another UMI with a larger family size.

In one embodiment, this disclosure provides a kit for labeling and amplifying each strand of at least 1 target genomic region with an oligonucleotide barcode sequence by polymerase chain reaction, the kit comprising: (a) a DNA polymerase; (b) dNTPs; (c) at least one Blocker Displacement Amplification (BDA) forward primer; (d) at least one BDA blocker; (e) at least one universal forward primer; (f) at least one universal reverse primer; and (g) at least one oligonucleotide comprising a Unique Molecular Identifier. In some embodiments, a kit further comprises a DNA polymerase buffer, nuclease-free water, or both.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Experimental workflow for one implementation of Quantitative Blocker Displacement Amplification (QBDA).

FIGS. 2A-2B. (FIG. 2A) Data analysis workflow for QBDA followed by next-generation sequencing. It is optional to remove families with UMI sequences similar to another UMI with a larger family size. This step aims to reduce the number of false UMI families that arise from polymerase error in a UMI sequence. (FIG. 2B) Reasons for using WT veto to call a UMI family genotype when a blocker is added in the protocol. Random mutations generated by polymerase error in early cycles will be enriched during BDA, so that a family originated from a WT molecule may have a majority of the reads as variant sequences. To solve this issue, a family is called as WT, if WT reads in the family are more than a percentage threshold (Pwt). Pwt is in the range of 0.01%-50%.

FIG. 3. Experimental workflow for QBDA with blocker(s) for non-specific target(s) to reduce non-specific amplification on pseudogene.

### DETAILED DESCRIPTION

The present disclosure provides methods of quantitative blocker displacement amplification (QBDA) sequencing for labeling each strand in targeted genomic regions of an original DNA sample with an oligonucleotide barcode sequence, and selective PCR amplification of DNA sequence variants across the targeted regions for quantitation. The amplified DNA can be analyzed by next-generation sequencing. The methods allow rare mutation quantitation and profiling using low-depth NGS. While the previously disclosed Blocker Displacement Amplification (BDA) methods require laborious calibration curves for quantitation, calibration-free quantitation is achieved in QBDA by using molecular barcodes.

This technology allows rare mutation quantitation and profiling using low-depth next-generation sequencing (NGS). A non-limiting SNP panel has been designed to validate the technology. In addition, also as a non-limiting example, a QBDA panel was designed to cover melanoma hot spot mutation sites and demonstrated the quantitation accuracy and clinical utility.

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents, published patent applications, and non-patent publications, are incorporated herein by reference in their entirety.

Any composition provided herein is specifically envisioned for use with any applicable method provided herein.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

When a range of numbers is provided herein, the range is understood to inclusive of the edges of the range as well as any number between the defined edges of the range. For example, “between 1 and 10” includes any number between 1 and 10, as well as the number 1 and the number 10.

### I. Definitions

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

“Incorporating,” as used herein, means becoming part of a nucleic acid polymer.

The term “in the absence of exogenous manipulation” as used herein refers to there being modification of a nucleic acid molecule without changing the solution in which the nucleic acid molecule is being modified. In specific embodiments, it occurs in the absence of the hand of man or in the absence of a machine that changes solution conditions, which may also be referred to as buffer conditions. However, changes in temperature may occur during the modification.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.” In addition, the term “adaptor” can indicate a linear adaptor (either single stranded or double stranded) or a stem-loop adaptor. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A “nucleic acid molecule” or “nucleic acid target molecule” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bi sulfite conversion, methylation/demethylation), extension, amplification (e.g., PCR, isothermal, etc.), etc.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization. In certain embodiments, a “complementary” nucleic acid contains at least one sequence in which 70%, 71%, 72%, 73%, 74%, 75%, 76%, 77%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, to 100%, and any range therein, of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

The term “degenerate” as used herein refers to a nucleotide or series of nucleotides wherein the identity can be selected from a variety of choices of nucleotides, as opposed to a defined sequence. In specific embodiments, there can be a choice from two or more different nucleotides. In further specific embodiments, the selection of a nucleotide at one particular position comprises selection from only purines, only pyrimidines, or from non-pairing purines and pyrimidines.

As used herein, the term “blocker oligonucleotide” refers to at least one continuous strand of from about 12 to about 100 nucleotides in length and if so indicated herein, may further include a functional group or nucleotide sequence at its 3′ end that prevents enzymatic extension during an amplification process such as polymerase chain reaction.

As used herein, the term “primer oligonucleotide” refers to a molecule comprising at least one continuous strand of from about 12 to about 100 nucleotides in length and sufficient to permit enzymatic extension during an amplification process such as polymerase chain reaction.

As used herein, the term “target-neutral subsequence” refers to a sequence of nucleotides that is complementary to a sequence in both a target nucleic acid and a variant nucleic acid. For example, a desired nucleic acid sequence to be targeted for amplification (target nucleic acid) may exist in a sample with a nucleic acid molecule having a predominantly homologous sequence with the target nucleic acid with the exception of a variable region (variant nucleic acid), such variable region in some instance being only a single nucleotide difference from the target nucleic acid. In this example, the target-neutral subsequence is complementary to at least a portion of the homologous sequence shared between the two nucleic acids, but not the variable region. Thus, as used herein, the term “blocker variable subsequence” refers to a nucleotide sequence of a blocker oligonucleotide which is complementary to the variable region of the variant nucleic.

As used herein, the term “overlapping subsequence” refers to a nucleotide sequence of at least 5 nucleotides of a primer oligonucleotide that is homologous with a portion of the blocker oligonucleotide sequence used in a composition as described herein. The overlapping subsequence of the primer oligonucleotide may be homologous to any portion of the target-neutral subsequence of the blocker oligonucleotide, whether 5′ or 3′ of the blocker variable subsequence. Thus, the term “non-overlapping subsequence” refers to the sequence of a primer oligonucleotide that is not the overlapping subsequence.

As used herein, the term “target sequence” or a “target genomic region” refers to the nucleotide sequence of a nucleic acid that comprises a desired allele, such as a single nucleotide polymorphism, to be amplified, identified, or otherwise isolated. As used herein, the term “variant sequence” refers to the nucleotide sequence of a nucleic acid that does not comprise the desired allele. For example, in some instances, the variant sequence comprises the wild-type allele whereas the target sequence comprises the mutant allele. Thus, in some instance, the variant sequence and the target sequence are derived from a common locus in a genome such that the sequences of each may be substantially homologous except for a region comprising the desired allele, nucleotide or group or nucleotides that varies between the two. In an aspect, a variant sequence comprises a single nucleotide polymorphism (SNP) as compared to a wild-type sequence or allele. In an aspect, a variant sequence comprises an insertion of at least one nucleotide as compared to a wild-type sequence or allele. In an aspect, a variant sequence comprises a deletion of at least one nucleotide as compared to a wild-type sequence or allele. In an aspect, a variant sequence comprises an inversion of at least two nucleotides as compared to a wild-type sequence or allele.

In some embodiments, a method provided herein labels and amplifies each strand of between 1 and 25,000, between 1 and 20,000, between 1 and 15,000, between 1 and 10,000, between 1 and 7500, between 1 and 5000, between 1 and 2500, between 1 and 1000, between 1 and 750, between 1 and 500, between 1 and 250, between 1 and 100, between 1 and 75, between 1 and 50, between 1 and 25, between 10 and 100, between 10 and 75, between 10 and 50, between 50 and 100, between 100 and 10,000, or between 1000 and 10,000 target genomic regions. In some embodiments, a method provided herein labels and amplifies each strand of at least 1, 2, 3, 4, 5, 6, 7, 8, 9, 10, 20, 30, 40, 50, 60, 70, 80, 90, 100, 250, 500, 750, 1000, 2500, 5000, 7500, 10,000, or 15,000 target genomic regions.

Non-limiting examples of target sequences from the human genome include AKT1, ALK, APC, AR, ATM, BRAF, CCND1, CDK4, CDKN2A, CHEK2, CTNNB1, DDR2, EGFR, ERBB2, ERBB3, ERBB4, ESR1, EZH2, FBXW7, FGFR1, FGFR2, FGFR3, FGFR4, FLT3, FOXL2, GNA11, GNAQ, GNAS, HRAS, IDH1, JAK1, JAK2, JAK3, KIT, KRAS, MAP2K1, MAP2K2, MET, MLH1, MPL, MTOR, MYC, MYCN, MYD88, NPM1, NRAS, PDGFRA, PIK3CA, PTEN, PTPN11, RAF1, RB1, RET, ROS1, SF3B1, SMAD4, SMARCB1, SMO, STK11, and TP53. In an aspect, a target sequence is selected from the group consisting of AKT1, ALK, APC, AR, ATM, BRAF, CCND1, CDK4, CDKN2A, CHEK2, CTNNB1, DDR2, EGFR, ERBB2, ERBB3, ERBB4, ESR1, EZH2, FBXW7, FGFR1, FGFR2, FGFR3, FGFR4, FLT3, FOXL2, GNA11, GNAQ, GNAS, HRAS, IDH1, JAK1, JAK2, JAK3, KIT, KRAS, MAP2K1, MAP2K2, MET, MLH1, MPL, MTOR, MYC, MYCN, MYD88, NPM1, NRAS, PDGFRA, PIK3CA, PTEN, PTPN11, RAF1, RB1, RET, ROS1, SF3B1, SMAD4, SMARCB1, SMO, STK11, and TP53.

“Sample” means a material obtained or isolated from a fresh or preserved biological sample or synthetically created source that contains nucleic acids of interest. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. In an aspect, a sample is obtained from a human. In an aspect, a sample is obtained from an animal. In an aspect, a sample is obtained from a plant. In an aspect, a sample is obtained from a fungus. In an aspect, a sample is obtained from a protozoan. In an aspect, a sample is obtained from a bacteria. In an aspect, a sample is obtained from a virus. In an aspect, an animal is selected from the group consisting of a mammal, a fish, a bird, a lizard, an amphibian, and an invertebrate. In an aspect, a mammal is selected from the group consisting of a non-human primate, a rodent, a marsupial, a lagomorph, a feline, a canine, and an ungulate.

In an aspect, a sample comprises genomic DNA (gDNA). In an aspect, a sample comprises formalin-fixed paraffin-embedded DNA (FFPE DNA). In an aspect, a sample comprises circulating free DNA (cfDNA). A sample that comprises DNA can be referred to as a “DNA sample.”

In an aspect, a sample comprises at least 10 ng, 15 ng, 20 ng, 25 ng, 30 ng, 40 ng, 50 ng, 60 ng, 70 ng, 80 ng, 90 ng, or 100 ng of nucleic acids. In an aspect, a sample comprises at least 10 ng, 15 ng, 20 ng, 25 ng, 30 ng, 40 ng, 50 ng, 60 ng, 70 ng, 80 ng, 90 ng, or 100 ng of DNA. In an aspect, a sample comprises at least 10 ng, 15 ng, 20 ng, 25 ng, 30 ng, 40 ng, 50 ng, 60 ng, 70 ng, 80 ng, 90 ng, or 100 ng of gDNA. In an aspect, a sample comprises at least 10 ng, 15 ng, 20 ng, 25 ng, 30 ng, 40 ng, 50 ng, 60 ng, 70 ng, 80 ng, 90 ng, or 100 ng of FFPE DNA. In an aspect, a sample comprises at least 10 ng, 15 ng, 20 ng, 25 ng, 30 ng, 40 ng, 50 ng, 60 ng, 70 ng, 80 ng, 90 ng, or 100 ng of cfDNA.

In an aspect, a sample comprises between 5 ng and 100 ng, between 5 ng and 75 ng, between 5 ng and 50 ng, between 5 ng and 40 ng, between 5 ng and 30 ng, between 5 ng and 20 ng, or between 5 ng and 10 ng of nucleic acids. In an aspect, a sample comprises between 5 ng and 100 ng, between 5 ng and 75 ng, between 5 ng and 50 ng, between 5 ng and 40 ng, between 5 ng and 30 ng, between 5 ng and 20 ng, or between 5 ng and 10 ng of DNA. In an aspect, a sample comprises between 5 ng and 100 ng, between 5 ng and 75 ng, between 5 ng and 50 ng, between 5 ng and 40 ng, between 5 ng and 30 ng, between 5 ng and 20 ng, or between 5 ng and 10 ng of gDNA. In an aspect, a sample comprises between 5 ng and 100 ng, between 5 ng and 75 ng, between 5 ng and 50 ng, between 5 ng and 40 ng, between 5 ng and 30 ng, between 5 ng and 20 ng, or between 5 ng and 10 ng of FFPE DNA. In an aspect, a sample comprises between 5 ng and 100 ng, between 5 ng and 75 ng, between 5 ng and 50 ng, between 5 ng and 40 ng, between 5 ng and 30 ng, between 5 ng and 20 ng, or between 5 ng and 10 ng of cfDNA.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

### II. Quantitative Blocker Displacement Amplification (QBDA) Library Preparation Workflow

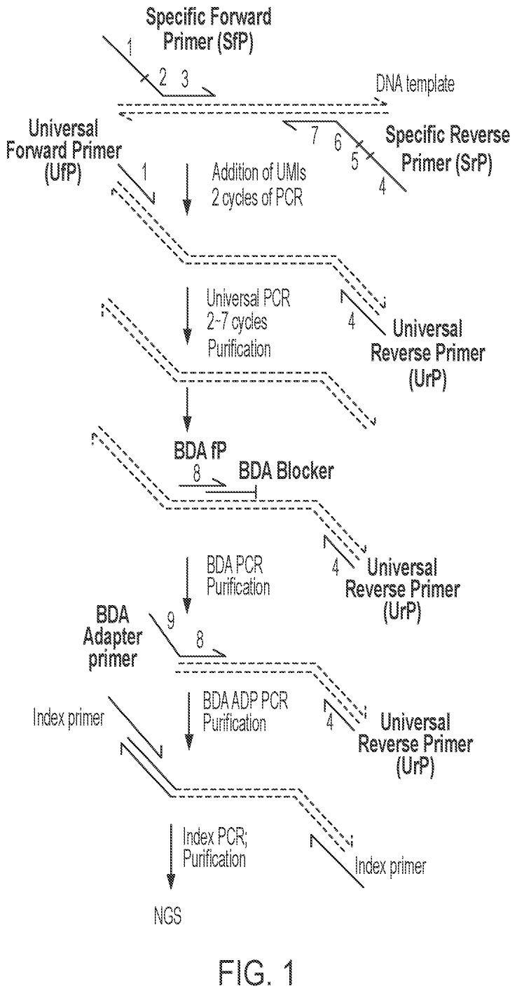

A schematic of the QASeq NGS library preparation workflow is shown in FIG. 1. The DNA sample is quantified by Qubit or qPCR to determine the input amount. For human genomic DNA, 1 ng DNA is considered as about 290 haploid genomic equivalents (or 580 strands).

For each amplification step, all types of DNA polymerases and PCR super mixes can be used, preferably using the standard annealing, extension, and denaturation temperature for the specific polymerase used, unless otherwise noted.

A. UMI Addition

First, a UMI addition step is performed. The DNA sample is mixed with a specific forward primer (SfP), a specific reverse primer (SrP), DNA polymerase, dNTPs, and PCR buffer. Two PCR cycles with a long extension time (about 30 min) are performed for addition of UMI on all target regions; each strand in one DNA molecule will carry a different UM″ A long extension time may be about 10 minutes (min), 11 min, 12 min, 13 min, 14 min, 15 min, 16 min, 17 min, 18 min, 19 min, 20 min, 21 min, 22 min, 23 min, 24 min, 25 min, 26 min, 27 min, 28 min, 29 min, 30 min, 31 min, 32 min, 33 min, 34 min, 35 min, 36 min, 37 min, 38 min, 39 min, 40 min, 41 min, 42 min, 43 min, 44 min, or 45 min, and preferably about 30 min. A long extension time may be within a range of about 10 min to about 45 min, about 10 min to about 40 min, about 10 min to about 35 min, about 10 min to about 30 min, about 15 min to about 45 min, about 15 min to about 40 min, about 15 min to about 35 min, about 15 min to about 30 min, about 15 min to about 25 min, about 20 min to about 45 min, about 20 min to about 40 min, about 20 min to about 35 min, about 20 min to about 30 min, about 25 min to about 45 min, about 25 min to about 40 min, about 25 min to about 35 min, or any range or value derivable therein.

The specific forward primer (SfP) comprises, from 5′ to 3′, regions 1, 2, and 3. Region 3 is the template-binding region; region 2 is an optional spacer region (typically 0 to 15 nucleotides (nt)) added for uniform amplification of different loci; region 1 is a universal primer binding site. SrP comprises, from 5′ to 3′, regions 4, 5, 6, and 7. Region 7 is the template-binding region; region 6 is an optional spacer region (typically 0 to 15 nt) added for uniform amplification of different loci; region 5 is the UMI region; region 4 is a universal primer binding site. Whole human genome nucleotide sequences can be searched to ensure that the primers are not prone to nonspecific amplification of non-target regions.

The concept of UMI is to give every original DNA molecule a different DNA sequence as a “barcode,” so that the origin of each NGS read can be tracked based on the barcode sequence. Given enough NGS reads, the number of unique UMIs found in the NGS output can reflect the number of original DNA molecules. Labeling each original molecule uniquely is achieved by using a large number of different UMI sequences; for example, using 10⁹ different UMI sequences for 100,000 original molecules will generate <0.006% molecules carrying repeated UMIs.

In an aspect, a UMI sequence comprises at least 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 nucleotides. In an aspect, a UMI comprises between 7 and 30, between 5 and 40, between 15 and 25, or between 10 and 20 nucleotides.

DNA sequences containing degenerate bases, such as poly(N) (i.e., a mix of A, T, C, or G at each position), are often used as UMI sequences. In QBDA, poly(H) (i.e., as mix of A, T, or C at each position) is used as the UMI because it has weaker cross-binding energy compared to poly(N) or a mix of S (C or G) and W (A or T) bases. (H)₂₀ contains 3.5×10⁹ different sequences, which is enough for 100,000 molecules as input; (H)₁₅ contains 1.4×10⁷ different sequences, which is enough for 6,000 molecules as input.

In an aspect, a UMI sequence comprises one or more degenerate nucleotides (or bases). Non-limiting examples of degenerate nucleotides include a C, G, or T nucleotide (B); an A, G, or T nucleotide (D); an A, C, or T nucleotide (H); a G or T nucleotide (K); an A or C nucleotide (M); any nucleotide (N); an A or G nucleotide (R); a G or C nucleotide (S); an A, C, or G nucleotide (V); an A or T nucleotide (W), and a C or T nucleotide (Y). In an aspect, each degenerate nucleotide in a UMI sequence is selected from the group consisting of N, B, D, H, V, S, W, Y, R, M, and K. In an aspect, every degenerate nucleotide of a UMI sequence is an H.

In some instances, it may be desirable to include a reverse blocker during UMI addition to prevent the addition of UMIs to a similar, but unwanted sequence, such as a pseudogene sequence. In this case, a reverse blocker can be designed and used according to the design criteria provided regarding the BDA blocker design for the Blocker Displacement Amplification step.

B. Universal Amplification

Second, a universal amplification step is performed. In order to amplify the molecules, and to avoid sample loss during purification while preventing addition of multiple UMIs onto the same original molecule, the annealing temperature is raised by about 0.01° C., about 0.1° C., about 1° C., about 2° C., about 3° C., about 4° C., about 5° C., about 6° C., about 7° C., about 8° C., about 9° C., or about 10° C. and about 2 cycles, about 3 cycles, about 4 cycles, about 5 cycles, about 6 cycles, about 7 cycles, about 8 cycles, about 9 cycles, or about 10 cycles are performed using Universal forward primer (UfP) and Universal reverse primer (UrP) with a short extension time. Preferably, the annealing temperature is raised about 8° C. Preferably, about 7 cycles are performed. A short extension time may be about 10 seconds (sec), 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, or 45 sec, and preferably about 30 sec. A short extension time may be within a range of about 10 sec to about 45 sec, about 10 sec to about 40 sec, about 10 sec to about 35 sec, about 10 sec to about 30 sec, about 15 sec to about 45 sec, about 15 sec to about 40 sec, about 15 sec to about 35 sec, about 15 sec to about 30 sec, about 15 sec to about 25 sec, about 20 sec to about 45 sec, about 20 sec to about 40 sec, about 20 sec to about 35 sec, about 20 sec to about 30 sec, about 25 sec to about 45 sec, about 25 sec to about 40 sec, about 25 sec to about 35 sec, or any range or value derivable therein.

In an aspect, an annealing temperature is raised by between 0.01° C. and 15° C., between 0.01° C. and 10° C., between 0.01° C. and 8° C., between 0.01° C. and 5° C., between 0.01° C. and 2° C. or between 0.01° C. and 1° C. between successive PCR amplifications in methods provided herein. In an aspect, an annealing temperature is the same (e.g., not changed) between successive PCR amplifications in methods provided herein. In an aspect, a melting temperature is raised by between 0.01° C. and 15° C., between 0.01° C. and 10° C., between 0.01° C. and 8° C., between 0.01° C. and 5° C., between 0.01° C. and 2° C. or between 0.01° C. and 1° C. between successive PCR amplifications in methods provided herein. In an aspect, a melting temperature is the same (e.g., not changed) between successive PCR amplifications in methods provided herein.

Addition of UfP and UrP into the reaction is an open-tube step on the thermocycler. Each QBDA panel only needs one Universal Forward Primer (UfP) and one Universal Reverse Primer (UrP). UfP comprises region 1, and UrP comprises region 4; there can be additional bases at the 5′-end of region 1 or region 4 in UfP or UrP. In some embodiments, the 5′-end of region 1 of the UfP can comprise at least 1, 2, 3, 4, 5, 6, 7, 8, 9, 10, 11, 12, 13, 14, 15, 20, or 25 additional bases. In some embodiments, the 5′-end of region 1 of the UrP can comprise at least 1, 2, 3, 4, 5, 6, 7, 8, 9, 10, 11, 12, 13, 14, 15, 20, or 25 additional bases. In some embodiments, the 5′-end of region 4 of the UfP can comprise at least 1, 2, 3, 4, 5, 6, 7, 8, 9, 10, 11, 12, 13, 14, 15, 20, or 25 additional bases. In some embodiments, the 5′-end of region 4 of the UrP can comprise at least 1, 2, 3, 4, 5, 6, 7, 8, 9, 10, 11, 12, 13, 14, 15, 20, or 25 additional bases.

Following the universal amplification step, purification is carried out to remove single-stranded primers, including SfP, SrP, UfP, and UrP. In some embodiments, purification involves a method selected from the group consisting of using SPRI magnetic beads, columns, or enzymatic digestion.

C. Blocker Displacement Amplification (BDA)

Third, blocker displacement amplification (BDA) is performed. BDA forward primer, BDA blocker, DNA polymerase, dNTPs, and PCR buffer are mixed with the purified PCR product for BDA amplification. The BDA forward primer anneals to a genomic region that is closer to the region that binds to SfP than the region that binds to SrP. In some embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 cycles of BDA amplification under conditions sufficient to achieve nucleic acid amplification are performed, then the PCR amplification product is purified by SPRI magnetic beads or columns. In some embodiments, between 2 and 30, between 2 and 25, between 2 and 23, between 2 and 20, between 2 and 15, between 4 and 30, between 4 and 25, between 4 and 23, between 4 and 20, between 4 and 15, between 6 and 30, between 6 and 25, between 6 and 23, between 6 and 20, between 6 and 15, between 8 and 30, between 8 and 25, between 8 and 23, between 8 and 20, between 8 and 15, between 10 and 30, between 10 and 25, between 10 and 23, between 10 and 20, between 10 and 15, or between 2 and 40 cycles of BDA amplification under conditions sufficient to achieve nucleic acid amplification are performed. In some embodiments, a PCR amplification product is purified by SPRI magnetic beads or columns. Preferably, between 10 and 23 cycles of BDA amplification under conditions sufficient to achieve nucleic acid amplification are performed, then the PCR reaction mixture is purified by SPRI magnetic beads or columns.

The BDA blocker may include a first sequence having a target-neutral subsequence and a blocker variable subsequence. In some aspects, the variable subsequence includes at least one nucleotide, at least two nucleotides, at least three nucleotides, at least four nucleotides, or at least five nucleotides. However, in some instances, the BDA blocker may not include the blocker variable subsequence if the target nucleic acid to be detected is for the detection of an insertion. The BDA blocker variable subsequence is flanked on its 3′ and 5′ ends by the target-neutral subsequence and is continuous with the target-neutral subsequence. The BDA blocker may comprise a functional group or a non-complementary sequence region at or near the 3′ end, which prevents enzymatic extension and/or 3′->5′ exonuclease activity by error-correct DNA polymerases. In an instance, the functional group of the blocker oligonucleotide is selected from, but is not limited to, the group consisting of a 3-carbon spacer or a dideoxynucleotide.

The BDA forward primer is sufficient to induce enzymatic extension of a template nucleic acid that is not bound by a BDA blocker. The 3′ end of the BDA forward primer includes a sequence that overlaps with the 5′ end of the BDA blocker. The portion of the BDA blocker that overlaps with the 3′ end of the BDA forward primer consists only of target-neutral subsequence, thus the BDA forward primer may not include any sequence homologous with the blocker variable subsequence.

The sequences of the BDA forward primer and BDA blocker may be rationally designed based on the thermodynamics of their hybridization to the target nucleic acid sequence (e.g., the sequence whose amplification or detection is desired, e.g., a SNP, an insertion, a deletion, or any other mutation) and the variant nucleic acid sequence (e.g., the sequence whose amplification is sought to be suppressed, e.g., a wild-type sequence). In some embodiments, the BDA blocker is present in a significantly higher concentration than the BDA forward primer, so that the preponderance of the target and the variant nucleic acid sequences bind to BDA blocker before binding to primer. The BDA forward primer binds transiently to the BDA blocker-target or BDA blocker-variant molecules and possesses a probability for displacing the BDA blocker in binding to the target or variant. Because the BDA blocker sequence is specific to the variant target, its displacement from the variant is less thermodynamically favorable than its displacement from the target. Thus, the non-allele-specific BDA forward primer amplifies the target sequence with higher yield/efficiency than it amplifies the variant sequence. The non-allele-specific nature of the BDA forward primer means that spuriously amplified variant sequence bears the variant allele, rather than the target allele, so that subsequent amplification cycles also exhibit amplification bias in favor of the target.

In some aspects, a BDA blocker contains a 3′ sequence or modification that prevents extension by a DNA polymerase. In an aspect, a modification is a terminator. In an aspect, a terminator is selected from the group consisting of a three-carbon (C3) spacer and DXXDM, where D is a match between the blocker sequence and the template nucleic acid molecule sequence, X is a mismatch between the blocker sequence and the template nucleic acid molecule sequence, and M is a C3 spacer. Additional terminators known in the art are also suitable for use. A non-limiting example of an additional terminator is a dideoxynucleotide.

In some aspects, the BDA blocker and the BDA forward primer may be designed such that the binding of each oligonucleotide meets certain standard free energy of hybridization conditions. For example, the standard free energy of hybridization of the BDA forward primer to the template nucleic acid (ΔG°_{PT}) and the standard free energy of hybridization of the BDA blocker to the template nucleic acid having the target sequence (ΔG°_{BT}) satisfies the following condition:

+2 kcal/mol≥ΔG°_{PT}−ΔG°_{BT}≥−8 kcal/mol.

In some aspects, BDA blocker and the BDA forward primer may be designed such that ΔG°_{PT}−ΔG°_{BT} is between about +3 kcal/mol and about −10 kcal/mol, about +3 kcal/mol and about −9 kcal/mol, about +3 kcal/mol and about −8 kcal/mol, about +3 kcal/mol and about −7 kcal/mol, about +3 kcal/mol and about −6 kcal/mol, about +3 kcal/mol and about −5 kcal/mol, about +3 kcal/mol and about −4 kcal/mol, about +3 kcal/mol and about −3 kcal/mol, about +3 kcal/mol and about −2 kcal/mol, about +3 kcal/mol and about −1 kcal/mol, about +3 kcal/mol and about 0 kcal/mol, about +3 kcal/mol and about +1 kcal/mol, about +2 kcal/mol and about −10 kcal/mol, about +2 kcal/mol and about −9 kcal/mol, about +2 kcal/mol and about −8 kcal/mol, about +2 kcal/mol and about −7 kcal/mol, about +2 kcal/mol and about −6 kcal/mol, about +2 kcal/mol and about −5 kcal/mol, about +2 kcal/mol and about −4 kcal/mol, about +2 kcal/mol and about −3 kcal/mol, about +2 kcal/mol and about −2 kcal/mol, about +2 kcal/mol and about −1 kcal/mol, about +2 kcal/mol and about 0 kcal/mol, about +1 kcal/mole and about −10 kcal/mol, about +1 kcal/mol and about −9 kcal/mol, about +1 kcal/mol and about −8 kcal/mol, about +1 kcal/mol and about −7 kcal/mol, about +1 kcal/mol and about −6 kcal/mol, about +1 kcal/mol and about −5 kcal/mol, about +1 kcal/mol and about −4 kcal/mol, about +1 kcal/mol and about −3 kcal/mol, about +1 kcal/mol and about −2 kcal/mol, about +1 kcal/mol and about −1 kcal/mol, about 0 kcal/mole and about −10 kcal/mol, about 0 kcal/mol and about −9 kcal/mol, about 0 kcal/mol and about −8 kcal/mol, about 0 kcal/mol and about −7 kcal/mol, about 0 kcal/mol and about −6 kcal/mol, about 0 kcal/mol and about −5 kcal/mol, about 0 kcal/mol and about −4 kcal/mol, about 0 kcal/mol and about −3 kcal/mol, about 0 kcal/mol and about −2 kcal/mol, about −1 kcal/mole and about −10 kcal/mol, about −1 kcal/mol and about −9 kcal/mol, about −1 kcal/mol and about −8 kcal/mol, about −1 kcal/mol and about −7 kcal/mol, about −1 kcal/mol and about −6 kcal/mol, about −1 kcal/mol and about −5 kcal/mol, about −1 kcal/mol and about −4 kcal/mol, about −1 kcal/mol and about −3 kcal/mol, about −2 kcal/mole and about −10 kcal/mol, about −2 kcal/mol and about −9 kcal/mol, about −2 kcal/mol and about −8 kcal/mol, about −2 kcal/mol and about −7 kcal/mol, about −2 kcal/mol and about −6 kcal/mol, about −2 kcal/mol and about −5 kcal/mol, about −2 kcal/mol and about −4 kcal/mol, or about −2 kcal/mol and about −3 kcal/mol. In some aspects, BDA blocker and the BDA forward primer may be designed such that ΔG°_{PT}−ΔG°_{BT} is preferably between about −1 kcal/mol and about −4 kcal/mol at approximately 50° C., approximately 55° C., approximately 60° C., approximately 65° C., or approximately 70° C. in a buffer suitable for PCR.

In some aspects, the BDA forward primer may be designed such that the portion of the primer that does hybridize with the BDA blocker binding site has a standard free energy of hybridization (ΔG°₃) that is between about −4 kcal/mol and about −12 kcal/mol, about −4 kcal/mol and about −11 kcal/mol, about −4 kcal/mol and about −10 kcal/mol, about −4 kcal/mol and about −9 kcal/mol, about −4 kcal/mol and about −8 kcal/mol, about −4 kcal/mol and about −7 kcal/mol, about −4 kcal/mol and about −6 kcal/mol, about −5 kcal/mol and about −12 kcal/mol, about −5 kcal/mol and about −11 kcal/mol, about −5 kcal/mol and about −10 kcal/mol, about −5 kcal/mol and about −9 kcal/mol, about −5 kcal/mol and about −8 kcal/mol, about −5 kcal/mol and about −7 kcal/mol, about −6 kcal/mol and about −12 kcal/mol, about −6 kcal/mol and about −11 kcal/mol, about −6 kcal/mol and about −10 kcal/mol, about −6 kcal/mol and about −9 kcal/mol, about −6 kcal/mol and about −8 kcal/mol, about −7 kcal/mol and about −12 kcal/mol, about −7 kcal/mol and about −11 kcal/mol, about −7 kcal/mol and about −10 kcal/mol, about −7 kcal/mol and about −9 kcal/mol, about −8 kcal/mol and about −12 kcal/mol, about −8 kcal/mol and about −11 kcal/mol, about −8 kcal/mol and about −10 kcal/mol, about −9 kcal/mol and about −12 kcal/mol, about −9 kcal/mol and about −11 kcal/mol, or about −10 kcal/mol and about −12 kcal/mol.

Methods for the calculation of ΔG° values from sequence are known in the art. There exist different conventions for calculating the ΔG° of different region interactions. WO2015/179339, which is incorporated herein by reference in its entirety, provides exemplary energy calculations based on the nearest neighbor model. The calculation of ΔG°_{PT}, ΔG°_{BT}, and ΔG°_{BV} from the primer sequence, blocker sequence, target sequence, variant sequence, operational temperature, and operational buffer conditions are known to those skilled in the art. The operational temperature may be about 20° C., about 25° C., about 30° C., about 35° C., about 40° C., about 45° C., about 50° C., about 55° C., about 60° C., about 65° C., or about 70° C. The operational buffer conditions may be buffer conditions suitable for PCR. Unless specifically described otherwise, the standard free energy of hybridization is calculated based on an annealing temperature of 60° C., double-stranded DNA, and a Na⁺ concentration of 0.18 M.

In some aspects, the BDA forward primer and BDA blocker may each, individually, be from about 12-100, about 12-90, about 12-80, about 12-70, about 12-60, about 12-50, about 12-40, about 12-30, about 15-100, about 15-90, about 15-80, about 15-70, about 15-60, about 15-50, about 15-40, about 15-30, about 20-100, about 20-90, about 20-80, about 20-70, about 20-60, about 20-50, about 20-40, or about 20-30 nucleotides in length. In some aspects, the BDA forward primer and BDA blocker may each, individually, be at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

In some aspects, the portion of the BDA forward primer that hybridizes to the BDA blocker binding site is between about 5-40 nucleotides, about 7-40, about 9-40, about 11-40, about 13-40, about 15-40, about 20-40, about 25-40, about 30-40, about 35-40, about 5-35, about 7-35, about 9-35, about 11-35, out 13-35, about 15-35, about 20-35, about 25-35, about 30-35, about 5-30, about 7-30, about 9-30, about 11-30, out 13-30, about 15-30, about 20-30, about 25-30, about 5-25, about 7-25, about 9-25, about 11-25, out 13-25, about 15-25, about 20-25, about 5-20, about 7-20, about 9-20, about 11-20, out 13-20, or about 15-20 nucleotides. In some aspects, the portion of the BDA forward primer that hybridizes to the BDA blocker binding site is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

In some aspects, the concentration of the BDA blocker is between about 2-10,000, about 2-9,000, about 2-8,000, about 2-7,000, about 2-6,000, about 2-5,000, about 2-4,000, about 2-3,000, about 2-2,000, about 2-1,000, about 2-900, about 2-800, about 2-700, about 2-600, about 2-500, about 2-400, about 2-300, about 2-200, about 2-150, about 2-100, about 2-90, about 2-80, about 2-70, about 2-60, about 2-50, about 2-40, about 2-30, about 2-20, about 2-10, about 4-10,000, about 4-9,000, about 4-8,000, about 4-7,000, about 4-6,000, about 4-5,000, about 4-4,000, about 4-3,000, about 4-2,000, about 4-1,000, about 4-900, about 4-800, about 4-700, about 4-600, about 4-500, about 4-400, about 4-300, about 4-200, about 4-150, about 4-100, about 4-90, about 4-80, about 4-70, about 4-60, about 4-50, about 4-40, about 4-30, about 4-20, about 4-10, about 6-10,000, about 6-9,000, about 6-8,000, about 6-7,000, about 6-6,000, about 6-5,000, about 6-4,000, about 6-3,000, about 6-2,000, about 6-1,000, about 6-900, about 6-800, about 6-700, about 6-600, about 6-500, about 6-400, about 6-300, about 6-200, about 6-150, about 6-100, about 6-90, about 6-80, about 6-70, about 6-60, about 6-50, about 6-40, about 6-30, about 6-20, about 6-10, about 10-10,000, about 10-9,000, about 10-8,000, about 10-7,000, about 10-6,000, about 10-5,000, about 10-4,000, about 10-3,000, about 10-2,000, about 10-1,000, about 10-900, about 10-800, about 10-700, about 10-600, about 10-500, about 10-400, about 10-300, about 10-200, about 10-150, about 10-100, about 10-90, about 10-80, about 10-70, about 10-60, about 10-50, about 10-40, about 10-30, about 10-20, about 20-10,000, about 20-9,000, about 20-8,000, about 20-7,000, about 20-6,000, about 20-5,000, about 20-4,000, about 20-3,000, about 20-2,000, about 20-1,000, about 20-900, about 20-800, about 20-700, about 20-600, about 20-500, about 20-400, about 20-300, about 20-200, about 20-150, about 20-100, about 20-90, about 20-80, about 20-70, about 20-60, about 20-50, about 20-40, about 20-30, about 40-10,000, about 40-9,000, about 40-8,000, about 40-7,000, about 40-6,000, about 40-5,000, about 40-4,000, about 40-3,000, about 40-2,000, about 40-1,000, about 40-900, about 40-800, about 40-700, about 40-600, about 40-500, about 40-400, about 40-300, about 40-200, about 40-150, about 40-100, about 40-90, about 40-80, about 40-70, about 40-60, or about 40-50 times greater than the concentration of the BDA forward primer. In some aspects, the concentration of the BDA blocker is at least 2, 5, 10, 20, 25, 50, 75, 100, 250, 500, 1000, 2500, 5000, 7500, or 10,000 times greater than the concentration of the BDA forward primer.

For multiplex BDA (mBDA) to simultaneously enrich potential target sequences at many groups of genetic loci, different BDA forward primers and BDA blockers are employed for each locus. These are all combined in solution simultaneously with the sample, a DNA polymerase, dNTPs, and buffers amenable for PCR. To prevent DNA-based inhibition of PCR, the total concentration of all oligo species can be kept under 50 micromolar. The length of the anneal/extend step of the PCR reaction is inversely proportional to the concentration of the lowest of the BDA forward primer species. To prevent excessively long protocols, it is recommended that all BDA forward primer concentrations be at least 100 picomolar. The concentration of each BDA blocker species should be at least 2× that of its corresponding BDA forward primer species.

In addition to the standard design principles of single-plex BDA described above, oligo design for multiplex BDA (mBDA) requires further consideration to prevent undesired “primer dimer” species. Algorithms for mBDA sequence design should penalize candidate sequence sets when they are predicted to exhibit nonselective binding interactions. See, for example, WO 2019/164885, which is incorporated herein by reference in its entirety.

D. Adapter Addition

Next, BDA adapter is added. BDA adaptor primer (comprising, e.g., Illumina adapter sequence and BDA forward primer sequence) and UrP are mixed with the purified PCR reaction mixture and amplified for at least 1 cycle. The amplification may be for at least 1, 2, 3, 4, 5, 6, 7, 8, 9, 10, 11, 12, 13, 14, 15, 16, 17, 18, 19 or 20 cycles. The adapter can also be added by enzymatic ligation reaction. Methods of using adaptor ligation to add additional sequences are described, e.g., in U.S. Pat. No. 7,803,550, which is incorporated by reference herein in its entirety.

E. Index PCR

Lastly, after another purification (for example, without being limiting, using SPRI magnetic beads or columns), standard NGS index PCR is performed; libraries are normalized and loaded onto an Illumina sequencer. The NGS libraries can be sequenced by Illumina sequencer (both single-read and paired-end) or other next-generation sequencers such as Ion Torrent.

### III. Quantitative Blocker Displacement Amplification (QBDA) Data Analysis Workflow

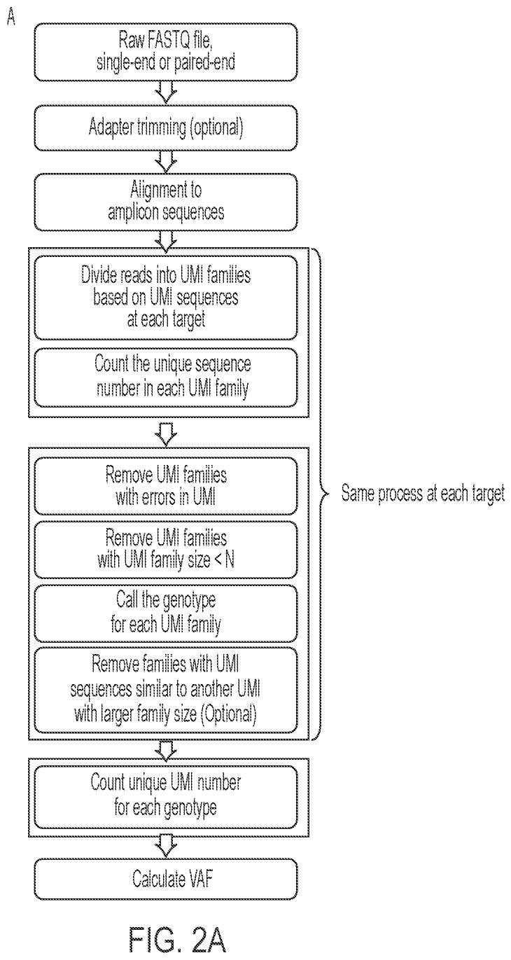

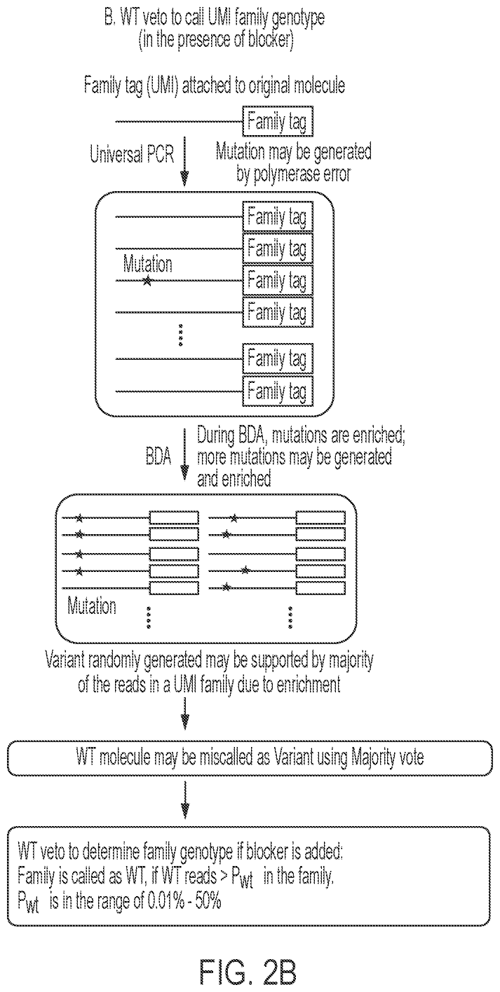

A schematic of the data analysis workflow for QBDA next-generation sequencing is shown in FIG. 2A. First, raw NGS reads are aligned to the amplicon regions; an optional adapter trimming can be performed before alignment. Unaligned reads are discarded, and the aligned reads are grouped by the genomic regions they aligned to.

Second, all the reads aligned to the same locus are divided by UMI sequences: reads carrying the same UMI are grouped as one UMI family. UMI family size is the number of reads carrying the same UMI, and unique UMI number is the total count of different UMI sequences at one locus.

Third, all UMI families that likely resulted from PCR polymerase errors or NGS sequencing errors are removed. A UMI sequence that is not consistent with designed UMI pattern (e.g., G bases found in the poly(H) UMI sequence) is an error and should be removed.

The UMI families with family sizes <Fₘᵢₙ will also be removed; Fₘᵢₙ may be 10, 9, 8, 7, 6, 5, 4, 3, 2, or 1. Fₘᵢₙ=4 is preferable in most cases. Optionally, Fₘᵢₙ is determined by the distribution of UMI family size. For example, Fₘᵢₙ may be the average of top 3 largest family sizes divided by 20; or Fₘᵢₙ may be the median family size divided by 10. Optionally, if two UMI sequences only differ by about 1-2 bases, the one with the smaller UMI family size is likely mutated from the other, and thus can be removed.

The genotype for each UMI family is then determined. If a sequence is supported by more than 50%, 55%, 60%, 70%, 75%, 80%, 85%, or 90%, and preferably 70%, of the reads in the UMI family, the sequence will be genotype for that family. If no sequence is supported by more than 50%, 55%, 60%, 70%, 75%, 80%, 85%, or 90%, and preferably 70%, of the reads, the UMI family is discarded. Furthermore, WT veto is applied to call UMI family genotype when blocker is added in the protocol (FIG. 2B). Random mutation in the BDA enrichment region may be generated by polymerase errors in early cycles, and will be enriched during BDA. In this case, a family that originated from a WT molecule may have a majority of the reads as variant sequences. To solve this issue, a family is called as WT if BDA blocker is added and WT reads in the family are more than a percentage threshold (P_{wt}). P_{wt} is in the range of 0.01%-50%, 0.01%-45%, 0.01%-40%, 0.01%-35%, 0.01%-30%, 0.01% −25%, 0.01%, −20%, 0.01%-15%, 0.01%-10%, 0.01%-5%, 0.1%-50%, 0.1%-45%, 0.1% −40%, 0.1%-35%, 0.1%-30%, 0.1%-25%, 0.1%, −20%, 0.1%-15%, 0.1%-10%, 0.1% −5%, 0.5%-50%, 0.1%-45%, 0.5%-40%, 0.5%-35%, 0.5%-30%, 0.5%-25%, 0.5%, −20%, 0.5%-15%, 0.5%-10%, 0.5%-5%, 1%-50%, 1%-45%, 1%-40%, 1%-35%, 1% −30%, 1%-25%, 1%, −20%, 1%-15%, 1%-10%, or 1%-5%. P_{wt} may be about 0.01%, 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%. In some embodiments, P_{wt} is at least 1%, 2%, 3%, 4%, 5%, 6%, 7%, 8%, 9%, 10%, 15%, 20%, 25%, 30%, 35%, 40%, 45%, or 50%.

The unique UMI number (N) for each mutation will be used for the calculation of VAF. VAF for a variant will be calculated as VAF=Nᵥₐᵣ/(Nᵢₙₚᵤₜ*Yield), where Nᵥₐᵣ is the unique UMI number for the variant sequence, Nᵢₙₚᵤₜ is the strand number of DNA input for QBDA, and Yield is the overall conversion yield of QDBA method. Conversion yield is calibrated by carrying out QBDA experiment without BDA blocker. For example, if the input molecule Nᵢₙₚᵤₜ=6,000 and observed unique UMI number is 4,000 when no blocker is added, the conversion yield=4,000/6,000=66.7%. The DNA input can be determined by Qubit or qPCR. For human genomic DNA, 1 ng DNA is considered as about 290 haploid genomic equivalence (or 580 strands). Thus, if 10 ng human gDNA is used as input, Nᵢₙₚᵤₜ=10*580=5,800. If needed, a qPCR experiment can be done (e.g., on FFPE samples) to accurately quantify the number of amplifiable DNA molecules.

### IV. Further Processing of Target Nucleic Acids

A. Amplification of DNA

A number of template-dependent processes are available to amplify the nucleic acids present in a given template sample. One of the best known amplification methods is the polymerase chain reaction (referred to as PCR′) which is described in detail in U.S. Pat. Nos. 4,683,195, 4,683,202, and 4,800,159 and in Innis et al., 1990, each of which is incorporated herein by reference in their entirety. Briefly, two synthetic oligonucleotide primers, which are complementary to two regions of the template DNA (one for each strand) to be amplified, are added to the template DNA (that need not be pure), in the presence of excess deoxynucleotides (dNTP's) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 50-60° C.) and a daughter strand extended from the primers (72° C.). As the daughter strands are created they act as templates in subsequent cycles. Thus, the template region between the two primers is amplified exponentially, rather than linearly.

B. Sequencing of DNA

Methods are also provided for the sequencing of the library of adaptor-linked fragments. Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit, and additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). Additional methods for next-generation sequencing methods, including various methods for library construction that may be used with embodiments of the present invention are described, e.g., in Pareek (2011) and Thudi (2012).

In particular aspects, the sequencing technologies used in the methods of the present disclosure include the HiSeg™ system (e.g., HiSeg™ 2000 and HiSeg™ 1000), the NextSeg™ 500, and the MiSeg™ system from Illumina, Inc. The HiSeg™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSeg™ system uses TruSeq™, Illumina's reversible terminator-based sequencing-by-synthesis.

Another example of a DNA sequencing technique that can be used in the methods of the present disclosure is 454 sequencing (Roche) (Margulies et al., 2005). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2009). Complete genomics' CGA Platform uses a novel strategy called combinatorial probe anchor ligation (cPAL) for sequencing. The process begins by hybridization between an anchor molecule and one of the unique adapters. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n+1, n+2, n+3, and n+4 positions.

### V. Kits

The technology described herein includes kits for quantitatively analyzing variant allele frequencies in a DNA sample. A “kit” refers to a combination of physical elements. For example, a kit may include, for example, one or more components such as nucleic acid primers, nucleic acid blockers, enzymes, reaction buffers, an instruction sheet, and other elements useful to practice the technology described herein. These physical elements can be arranged in any way suitable for carrying out the invention.

In an aspect, a kit provided herein comprises a DNA polymerase. In an aspect, a kit provided herein comprises a DNA polymerase buffer. In an aspect, a kit provided herein comprises dNTPs. In an aspect, a kit provided herein comprises nuclease-free water. In an aspect, a kit provided herein comprises a universal forward primer, a universal reverse primer, or both. In an aspect, a kit provided herein comprises at least one BDA forward primer. In an aspect, a kit provided herein comprises at least one BDA blocker. In an aspect, a kit provided herein comprises at least one oligonucleotide comprising a UMI. In an aspect, a kit provided herein comprises an nucleic acid molecule that serves as a positive control. In an aspect, a kit provided herein comprises an nucleic acid molecule that serves as a negative control.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow molded plastic containers into which the desired vials are retained. A kit will also include instructions for employing the kit components as well the use of any other reagent not included in the kit. Instructions may include variations that can be implemented.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—QBDA SNP Panel

A SNP panel was built to validate the performance of QBDA. Then SNP loci that are different in NA18562 and NA18537 were selected (Table 1). BDA has been shown to perform well on these 10 SNP loci. A QBDA panel was designed based on previous normal BDA design. The sequences of SfP, SrP, UfP, UrP, BDA fp, BDA blocker, and Adp_fp are provided in Table 2. Genomic DNA NA18562 was mixed with NA18537 to get mixture samples with NA18562 spike-in ratios of 1.0% and 0.1%. 30 ng of the 1.0% and 0.1% spike-in gDNA mixtures were used as QBDA input. NGS library preparation and data analysis were performed as described above.

Because the spike-in samples are made in bulk, the true VAF value for each of the 10 SNP loci is 1% or 0.1%. 30 ng gDNA corresponds to about 8,700 haploid copy and 17,400 strands. Thus, the expected molecule number for 1% and 0.1% spike-in should be 174 and 17, respectively. VAF is calculated as Observed Var Molecule Number/(17,400*Yield). As shown in the quantitation results summarized in Table 3, the calculated VAF is close to the expected true value for all the 10 targets. For 1% spike-in, the calculated VAF is in the range of 0.5%-1.7%, with an average of 1.2%. For 0.1% spike-in, the calculated VAF is in the range of 0.04%-0.21%, with an average of 0.09%. There are only 9 haploid copies of Variant in 30 ng gDNA with 0.1% spike-in ratio; so the Poisson sampling error makes the quantitation for 0.1% spike-in less accurate.

TABLE 1
Information for SNPs covered in the 10-plex SNP panel

| Target | | NA18537 | NA18562 |
|---|---|---|---|
| Number | SNP rs number | Genotype | Genotype |
| 1 | rs12478327 | C | A |
| 2 | rs2638145 | C | A |
| 3 | rs2170091 | A | C |
| 4 | rs2043583 | T | G |
| 5 | rs2246745 | T | A |
| 6 | rs1884444 | T | G |
| 7 | rs10833604 | C | T |
| 8 | rs17560702 | C | T |
| 9 | rs2215492 | C | T |
| 10 | rs2301720 | A | C |

TABLE 2
Oligonucleotide sequences used in the SNP panel

| | | |
|---|---|---|
| SfP_SNP1 | GGATATTCCTTTCTACTCTTTGACATCATCTATCATG | SEQ ID |
| | TTTTCATGGCAAACCCTAACATC | NO: 1 |
| SfP_SNP2 | GGATATTCCTTTCTACTCTTTGACATCATCTATCACA | SEQ ID |
| | AAGTACTGGCCTTTGCTGCTTA | NO: 2 |
| SfP_SNP3 | GGATATTCCTTTCTACTCTTTGACATCATCTATCACA | SEQ ID |
| | TGAGAGGGCTCTAAATAATCTCCTTC | NO: 3 |
| SfP_SNP4 | GGATATTCCTTTCTACTCTTTGACATCATCTATCACC | SEQ ID |
| | AACAGGTATTGATTCTTGAGGACTTA | NO: 4 |
| SfP_SNP5 | GGATATTCCTTTCTACTCTTTGACATCATCTATCACA | SEQ ID |
| | GTGTTTTCTTTATTTTTATGTCCAGGTCA | NO: 5 |
| SfP_SNP6 | GGATATTCCTTTCTACTCTTTGACATCATCTATCAAT | SEQ ID |
| | TTTTCATATTTTTTTTCCAGAGGGAAACAG | NO: 6 |
| SfP_SNP7 | GGATATTCCTTTCTACTCTTTGACATCATCTATCATC | SEQ ID |
| | TGCACCATGTTCCTCCAACTAT | NO: 7 |
| SfP_SNP8 | GGATATTCCTTTCTACTCTTTGACATCATCTATCATG | SEQ ID |
| | TG CTACTCTGACCAAGACAGAA | NO: 8 |
| SfP_SNP9 | GGATATTCCTTTCTACTCTTTGACATCATCTATCACT | SEQ ID |
| | GTGCAGACATAATGGCAGGA | NO: 9 |
| SfP_SNP10 | GGATATTCCTTTCTACTCTTTGACATCATCTATCACG | SEQ ID |
| | GAACGGTCGAGGCGAA | NO: 10 |
| Universal Forward | CCTATGGTAGTTAAATGTAGATTGGATATTCCTTTCT | SEQ ID |
| Primer | ACTCTTTGACATCATCT | NO: 11 |
| SrPSNP1 | AGACGTGTGCTCTTCCGATCTATCAHHHHHHHHHHHH | SEQ ID |
| | HHHGTGCAAGCTGGAGGCACT | NO: 12 |
| SrP_SNP2 | AGACGTGTGCTCTTCCGATCTATCAHHHHHHHHHHHH | SEQ ID |
| | HHHACAGGAAAAGAAACTAAAATTGTAGCCTT | NO: 13 |
| SrP_SNP3 | AGACGTGTGCTCTTCCGATCTATCAHHHHHHHHHHHH | SEQ ID |
| | HHHGAAGCCAGATCTCAAAGTGTCCT | NO: 14 |
| SrP_SNP4 | AGACGTGTGCTCTTCCGATCTATCAHHHHHHHHHHHH | SEQ ID |
| | HHHGTTATTGGGAATGCTATGAAAGAGACA | NO: 15 |
| SrP_SNP5 | AGACGTGTGCTCTTCCGATCTATCAHHHHHHHHHHHH | SEQ ID |
| | HHHGGCTGCGATGAGACAGGAA | NO: 16 |
| SrP_SNP6 | AGACGTGTGCTCTTCCGATCTATCAHHHHHHHHHHHH | SEQ ID |
| | HHHTGAAAGATAGCAATAGATACATAAAACAC GA | NO: 17 |
| SrP_SNP7 | AGACGTGTGCTCTTCCGATCTATCAHHHHHHHHHHHH | SEQ ID |
| | HHHCAATTTCCAAGACAGAAGCACTCC | NO: 18 |
| SrP_SNP8 | AGACGTGTGCTCTTCCGATCTATCAHHHHHHHHHHHH | SEQ ID |
| | HHHGGGGGAAAATGGTTTCTTAGGATGA | NO: 19 |
| SrP_SNP9 | AGACGTGTGCTCTTCCGATCTATCAHHHHHHHHHHHH | SEQ ID |
| | HHHAGCATGCCGCCCTTGG | NO: 20 |
| SrP_SNP10 | AGACGTGTGCTCTTCCGATCTATCAHHHHHHHHHHHH | SEQ ID |
| | HHHTCACAGGTCAAAATTATGAGTTCTTCG | NO: 21 |
| Universal Reverse | GACTGGAGTTCAGACGTGTGCTCTTCCGATCT | SEQ ID |
| Primer | | NO: 22 |
| Adp_fp_SNP1 | ACACGACGCTCTTCCGATCTACTTCTGCCAACATTCA | SEQ ID |
| | AATTCAGG | NO: 23 |
| Adp_fp_SNP2 | ACACGACGCTCTTCCGATCTGGATGGGACTCCAATGC | SEQ ID |
| | AAAACT | NO: 24 |
| Adp_fp_SNP3 | ACACGACGCTCTTCCGATCTCATCTTGCTCTTCATAG | SEQ ID |
| | ATAGCTTCAGA | NO: 25 |
| Adp_fp_SNP4 | ACACGACGCTCTTCCGATCTCCTGAATGTCAGTTTTG | SEQ ID |
| | TTAGAGCAAC | NO: 26 |
| Adp_fp_SNP5 | ACACGACGCTCTTCCGATCTCTCCTTGGAATCACCAA | SEQ ID |
| | GAAAGAT | NO: 27 |
| Adp_fp_SNP6 | ACACGACGCTCTTCCGATCTTTCCTGCTTCCAGACAT | SEQ ID |
| | GAATCA | NO: 28 |
| Adp_fp_SNP7 | ACACGACGCTCTTCCGATCTCTCTCTAGAGTGCAGAT | SEQ ID |
| | TGGTAGAA | NO: 29 |
| Adp_fp_SNP8 | ACACGACGCTCTTCCGATCTACATGTCCAAAGAGAGA | SEQ ID |
| | AGTCGTAG | NO: 30 |
| Adp_fp_SNP9 | ACACGACGCTCTTCCGATCTGCCCCAAAGGTTACCCC | SEQ ID |
| | ATG | NO: 31 |
| Adp_fp_SNP10 | ACACGACGCTCTTCCGATCTGTAGCCGCTTCTCTGTG | SEQ ID |
| | AGTT | NO: 32 |
| BDA_fp_SNP1 | ACTTCTGCCAACATTCAAATTCAGG | SEQ ID |
| | | NO: 33 |
| BDA_fp_SNP2 | GGATGGGACTCCAATGCAAAACT | SEQ ID |
| | | NO: 34 |
| BDA_fp_SNP3 | CATCTTGCTCTTCATAGATAGCTTCAGA | SEQ ID |
| | | NO: 35 |
| BDA_fp_SNP4 | CCTGAATGTCAGTTTTGTTAGAGCAAC | SEQ ID |
| | | NO: 36 |
| BDA_fp_SNP5 | CTCCTTGGAATCACCAACAAACAT | SEQ ID |
| | | NO: 37 |
| BDA_fp_SNP6 | TTCCTGCTTCCAGACATGAATCA | SEQ ID |
| | | NO: 38 |
| BDA_fp_SNP7 | CTCTCTAGAGTGCAGATTGGTAGAA | SEQ ID |
| | | NO: 39 |
| BDA_fp_SNP8 | ACATGTCCAAAGAGAGAAGTC GTAG | SEQ ID |
| | | NO: 40 |
| BDA_fp_SNP9 | GCCCCAAAGGTTACCCCATG | SEQ ID |
| | | NO: 41 |
| BDA_fp_SNP10 | GTAGCCGCTTCTCTGTGAGTT | SEQ ID |
| | | NO: 42 |
| BDA_blocker_SNP1 | TCAAATTCAGGTACCTTAGAGGGACAGCTAAA/ | SEQ ID |
| | ISPC3//ISPC3/CT | NO: 43 |
| BDA_blocker_SNP2 | AATGCAAAACTCAATGTATCAGTGTGAGGATGT/ | SEQ ID |
| | ISPC3//ISPC3/AT | NO: 44 |
| BDA_blocker_SNP3 | TAGCTTCAGAAACATTCCAGTGTATGTGCAG/ | SEQ ID |
| | ISPC3//ISPC3/GA | NO: 45 |
| BDA_blocker_SNP4 | GTTAGAGCAACTTTCCTTGATTCCCAGAGTAG/ | SEQ ID |
| | ISPC3//ISPC3/CT | NO: 46 |
| BDA_blocker_SNP5 | CAACAAACATGCCTTCTCCTTCTCCTGA/ISPC3// | SEQ ID |
| | ISPC3/AA | NO: 47 |
| BDA_blocker_SNP6 | ACATGAATCATGTCACTATTCAATGGGATGC/ | SEQ ID |
| | ISPC3//ISPC3/TT | NO: 48 |
| BDA_blocker_SNP7 | GATTGGTAGAAGACACTGATTGCATCTTCAA/ | SEQ ID |
| | ISPC3//ISPC3/GT | NO: 49 |
| BDA_blocker_SNP8 | GAAGTCGTAGCTATTCGGCAAAGGAAATG/ISPC3// | SEQ ID |
| | ISPC3/TT | NO: 50 |
| BDA_blocker_SNP9 | TACCCCATGTGTATCAAATGGTCAGCAAG/ISPC3// | SEQ ID |
| | ISPC3/TT | NO: 51 |
| BDA_blocker_SNP10 | CTGTGAGTTGGGAGCAAAGGAGCA/ISPC3// | SEQ ID |
| | ISPC3/AT | NO: 52 |

TABLE 3
Quantitation validation of a 10-plex SNP panel

| NA18562 | | | | | | | | | | | |
|---|---|---|---|---|---|---|---|---|---|---|---|
| spike- | | | | | | | | | | | |
| in % | | Tar1 | Tar2 | Tar3 | Tar4 | Tar5 | Tar6 | Tar7 | Tar8 | Tar9 | Tar10 |
| 1% | Obs. Var | 209 | 185 | 87 | 170 | 139 | 140 | 155 | 189 | 249 | 159 |
| | Mol | | | | | | | | | | |
| | Number | | | | | | | | | | |
| | Yield | 1.01 | 0.71 | 1.00 | 0.92 | 0.85 | 0.58 | 0.65 | 0.79 | 1.12 | 0.54 |
| | Calc. VAF | 1.2% | 1.5% | 0.5% | 1.1% | 0.9% | 1.4% | 1.4% | 1.4% | 1.3% | 1.7% |
| 0.10% | Obs. Var | 37 | 12 | 7 | 11 | 14 | 8 | 6 | 17 | 9 | 10 |
| | Mol | | | | | | | | | | |
| | Number | | | | | | | | | | |
| | Yield | 1.01 | 0.71 | 1.00 | 0.92 | 0.85 | 0.58 | 0.65 | 0.79 | 1.12 | 0.54 |
| | Calc. VAF | 0.21% | 0.10% | 0.04% | 0.07% | 0.09% | 0.08% | 0.05% | 0.12% | 0.05% | 0.11% |

Calc. VAF = Obs. Var Mol Number/(17,400*Yield)

### Example 2—QBDA Melanoma Mutation Hotspot Panel

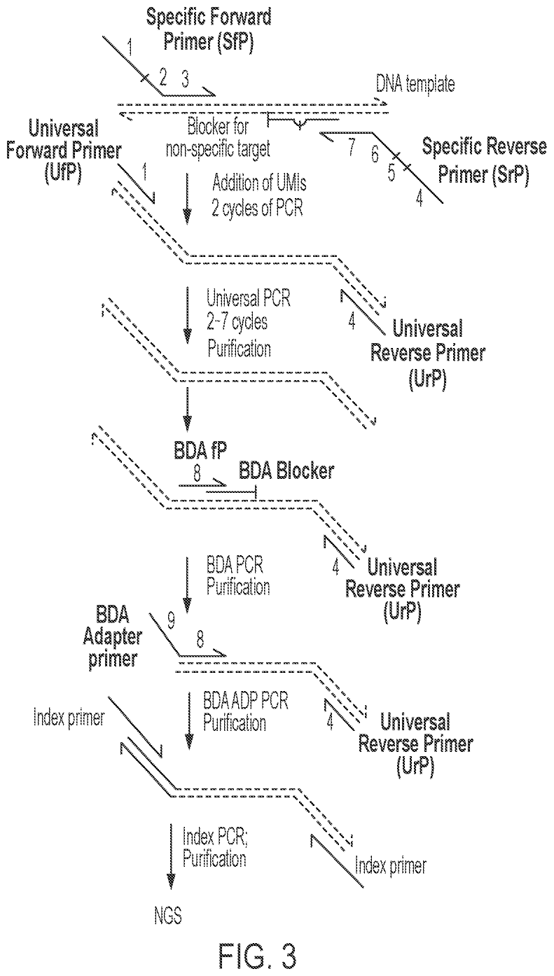

A 15-plex QBDA panel covering the hotspot mutations in melanoma was built and validated. The panel consists of 15 amplicons and covers 8 genes (MAP2K1, MAP2K2, AKT1, AKT3, NRAS, KRAS, PIK3CA, BRAF) and 22 hot spot amino acid mutation sites. There are 370 different mutations reported in COSMIC in the 22 mutation sites. The panel coverage information is summarized in Table 4. The oligonucleotide sequences used in the melanoma panel are provided in Table 5. Due to the presence of a pseudogene with very similar sequences in target 13 amplicon (PIK3CA), the experimental workflow was adjusted such that a reverse blocker was added together with the SfP and SrP to reduce non-specific amplification at the pseudogene (FIG. 3). The sequence corresponding to the pseudogene was also removed during data analysis.

gBlocks (sequence-verified, double-stranded DNA molecules) from IDT were added to NA18562 gDNA to make spike-in samples for panel validation. For each of the 15 enrichment regions, one pathogenic mutation reported in COSMIC was selected and the gBlock corresponding to the selected mutation was added. The gBlock information is summarized in Table 6. The selected 15 gBlocks were diluted and quantified by qPCR and then added into NA18562 to get approximately 1% spike-in. The mixture sample with 11,447 strands of NA18562 gDNA and about 1% spike-in gBlocks was used as input for the QBDA melanoma panel. VAF was calculated as Observed Var Molecule Number/11,447. As shown in the quantitation results summarized in Table 6, the calculated VAF was close to 1% and was close to the Variant reads frequency (VRF) obtained from another sequencing experiment with no blocker and without considering UMI.

Clinical tissue samples from melanoma patients or healthy people were also tested. The samples were purchased from commercial vendors. 20 ng Fresh Frozen (FF) or 40 ng Formalin-fixed, Paraffin-embedded (FFPE) tissue samples are used as input. For FF samples, 20 ng extracted gDNA corresponds to 11,600 strands, so VAF is calculated as Observed Var Molecule Number/(11,600*Yield). Because DNA extracted from FFPE is fragmented, qPCR experiment is performed on FFPE DNA samples to accurately quantify the number of amplifiable DNA molecules. As shown in Table 7, the quantitation results from QBDA were very close to the VRF obtained from independent sequencing experiment with no blocker and without considering UMI using the same samples. In 4 healthy volunteer gDNA samples (20 ng as input), no variants with VAF>0.1% were observed.

TABLE 4
Coverage of Melanoma QBDA panel

| | | | Covered | | |
|---|---|---|---|---|---|
| | | Enrichment Region | Hot Spot | Example of Covered | |
| Target | Gene | (GRCh38.p12) | AA Site | Mutation & COSMIC ID | |
| 1 | MAP2K1 | Chr15: 66435114- | 57 + 60 | K57E | COSM5369532 |
| | | 66435129 | | | |
| 2 | | Chr15: 66436814- | 121 + 124 | C121S | COSM555601 |
| | | 66436830 | | | |
| 3 | | Chr15: 66481788- | 203 | E203K | COSM232755 |
| | | 66481804 | | | |
| 4 | MAP2K2 | Chr19: 4117540- | 57 + 60 | F57V | COSM3534171 |
| | | 4117558 | | | |
| 5 | | Chr19: 4110573- | 125 + 126 | C125S | COSM5855815 |
| | | 4110588 | | | |
| 6 | | Chr19: 4101089- | 207 | E207K | COSM5574290 |
| | | 4101106 | | | |
| 7 | AKT1 | Chr14: 104776700- | 79 | Q79K | COSM159008 |
| | | 104776714 | | | |
| 8 | AKT3 | Chr1: 243695699- | 17 | E17K | COSM224779 |
| | | 243695726 | | | |
| 9 | NRAS | Chr1: 114716123- | 12 + 13 | G12S | COSM563 |
| | | 114716137 | | | |
| 10 | | Chr1: 114713894- | 61 | Q61K | COSM580 |
| | | 114713912 | | | |
| 11 | KRAS | Chr12: 25245346- | 12 + 13 | G12S | COSM517 |
| | | 25245358 | | | |
| 12 | | Chr12: 25227328- | 61 | Q61K | COSM87298 |
| | | 25227346 | | | |
| 13 | PIK3CA | Chr3: 179218291- | 542 + 545 | E542K | COSM760 |
| | | 179218309 | | | |
| 14 | | Chr3: 179234293- | 1047 | H1047Y | COSM774 |
| | | 179234307 | | | |
| 15 | BRAF | Chr7: 140753333- | 600 | V600E | COSM476 |
| | | 140753353 | | | |

TABLE 5
Oligonucleotide sequences used in the Melanoma panel

| | | |
|---|---|---|
| SfP_Mela1 | GGATATTCCTTTCTACTCTTTGACATCATCTA | SEQ ID |
| | TCAAAGCGCCTTGAGGCCTTT | NO: 53 |
| SfP_Mela2 | GGATATTCCTTTCTACTCTTTGACATCATCTA | SEQ ID |
| | TCAAAGGGAGCTGCAGGTTCTG | NO: 54 |
| SfP_Mela3 | GGATATTCCTTTCTACTCTTTGACATCATCTA | SEQ ID |
| | TCACTGCCCGCTGACCCC | NO: 55 |
| SfP_Mela4 | GGATATTCCTTTCTACTCTTTGACATCATCTA | SEQ ID |
| | TCATTGAGTTCGCCGACCTTGG | NO: 56 |
| SfP_Mela5 | GGATATTCCTTTCTACTCTTTGACATCATCTA | SEQ ID |
| | TCACGCGAGCTGCAGGTCC | NO: 57 |
| SfP_Mela6 | GGATATTCCTTTCTACTCTTTGACATCATCTA | SEQ ID |
| | TCATCCAACATCCTCGTGAACTCTAG | NO: 58 |
| SfP_Mela7 | GGATATTCCTTTCTACTCTTTGACATCATCTA | SEQ ID |
| | TGAGGCCCAACACCTTCATCATC | NO: 59 |
| SfP_Mela8 | GGATATTCCTTTCTACTCTTTGACATCATCTA | SEQ ID |
| | TCATGTCTTCAAAAGGAAGTATCTTGGC | NO: 60 |
| SfP_Mela9 | GGATATTCCTTTCTACTCTTTGACATCATCTA | SEQ ID |
| | TCAATTGTCAGTGCGCTTTTCCC | NO: 61 |
| SfP_Mela10 | GGATATTCCTTTCTACTCTTTGACATCATCTA | SEQ ID |
| | TCACTGTTTGTTGGACATACTGGATACA | NO: 62 |
| SfP_Mela11 | GGATATTCCTTTCTACTCTTTGACATCATCTA | SEQ ID |
| | TCAGTATCGTCAAGGCACTCTTGC | NO: 63 |
| SfP_Mela12 | GGATATTCCTTTCTACTCTTTGACATCATCTA | SEQ ID |
| | TCACTGTCTCTTGGATATTCTCGACAC | NO: 64 |
| SfP_Mela13 | GGATATTCCTTTCTACTCTTTGACATCATCTA | SEQ ID |
| | TCATCAAAG CAATTTCTACAC GAGATCC | NO: 65 |
| SfP_Mela14 | GGATATTCCTTTCTACTCTTTGACATCATCTA | SEQ ID |
| | TCAAGGCTTTGGAGTATTTCATGAAACAA | NO: 66 |
| SfP_Mela15 | GGATATTCCTTTCTACTCTTTGACATCATCTA | SEQ ID |
| | TCAACTGATGGGACCCACTCCAT | NO: 67 |
| Universal Forward | CCTATGGTAGTTAAATGTACATTGGATATTCC | SEQ ID |
| Primer | TTTCTACTCTTTGACATCATCT | NO: 68 |
| SrPMela1 | AGACGTGTGCTCTTCCGATCTATCAHHHHHHH | SEQ ID |
| | HHHHHHHHGGCTTGTGGGAGACCTTGA | NO: 69 |
| SrP_Mela2 | AGACGTGTGCTCTTCCGATCTATCAHHHHHHH | SEQ ID |
| | HHHHHHHHAGCCACCCAACTCTTAAGGC | NO: 70 |
| SrP_Mela3 | AGACGTGTGCTCTTCCGATCTATCAHHHHHHH | SEQ ID |
| | HHHHHHHHAGTTCCCTCCTTTTCTATTTTCTC | NO: 71 |
| | TTC | |
| SrP_Mela4 | AGACGTGTGCTCTTCCGATCTATCAHHHHHHH | SEQ ID |
| | HHHHHHHHAGTCTCCCTAGGTAGCTAACCC | NO: 72 |
| SrP_Mela5 | AGACGTGTGCTCTTCCGATCTATCAHHHHHHH | SEQ ID |
| | HHHHHHHHGGACGCACTCACCATGTGT | NO: 73 |
| SrP_Mela6 | AGACGTGTGCTCTTCCGATCTATCAHHHHHHH | SEQ ID |
| | HHHHHHHHGGGACTCACAGCCATGTAGG | NO: 74 |
| SrP_Mela7 | AGACGTGTGCTCTTCCGATCTATCAHHHHHHH | SEQ ID |
| | HHHHHHHHCCATCCCCGTGTCCCTC | NO: 75 |
| SrP_Mela8 | AGACGTGTGCTCTTCCGATCTATCAHHHHHHH | SEQ ID |
| | HHHHHHHHCCAGTGTTGTAGGACATATATTGT | NO: 76 |
| | ACC | |
| SrP_Mela9 | AGACGTGTGCTCTTCCGATCTATCAHHHHHHH | SEQ ID |
| | HHHHHHHHGCTTTAAAGTACTGTAGATGTGGC | NO: 77 |
| | TC | |
| SrP_Mela10 | AGACGTGTGCTCTTCCGATCTATCAHHHHHHH | SEQ ID |
| | HHHHHHHHGGTTAATATCCGCAAATGACTTGC | NO: 78 |
| SrP_Mela11 | AGACGTGTGCTCTTCCGATCTATCAHHHHHHH | SEQ ID |
| | HHHHHHHHTGTATTAACCTTATGTGTGACATG | NO: 79 |
| | TTCTAA | |
| SrP_Mela12 | AGACGTGTGCTCTTCCGATCTATCAHHHHHHH | SEQ ID |
| | HHHHHHHHTTATATTCAATTTAAACCCACCTA | NO: 80 |
| | TAATGGTG | |
| SrP_Mela13 | AGACGTGTGCTCTTCCGATCTATCAHHHHHHH | SEQ ID |
| | HHHHHHHHGGTATGGTAAAAACATGCTGAGAT | NO: 81 |
| | CA | |
| SrP_Mela14 | AGACGTGTGCTCTTCCGATCTATCAHHHHHHH | SEQ ID |
| | HHHHHHHHCAGTGCAGTGTGGAATCCAG | NO: 82 |
| SrP_Mela15 | AGACGTGTGCTCTTCCGATCTATCAHHHHHHH | SEQ ID |
| | HHHHHHHHTTAGTTAGTACACCTCAGATATAT | NO: 83 |
| | TTCTTCATG | |
| Universal Reverse | GACTGGAGTTCAGACGTGTGCTCTTCCGATCT | SEQ ID |
| Primer | | NO: 84 |
| Adp_fp_Mela1 | ACACGACGCTCTTCCGATCTCCTTGAGGCCTT | SEQ ID |
| | TCTTACCCA | NO: 85 |
| Adp_fp_Mela2 | ACACGACGCTCTTCCGATCTAGCTGCAGGTTC | SEQ ID |
| | TGCATGA | NO: 86 |
| Adp_fp_Mela3 | ACACGACGCTCTTCCGATCTCGCTGACCCCAA | SEQ ID |
| | AGTCACA | NO: 87 |
| Adp_fp_Mela4 | ACACGACGCTCTTCCGATCTTTCGCCGACCTT | SEQ ID |
| | GGCT | NO: 88 |
| Adp_fp_Mela5 | ACACGACGCTCTTCCGATCTGCTGCAGGTCCT | SEQ ID |
| | GCACG | NO: 89 |
| Adp_fp_Mela6 | ACACGACGCTCTTCCGATCTCATCCTCGTGAA | SEQ ID |
| | CTCTAGAGG | NO: 90 |
| Adp_fp_Mela7 | ACACGACGCTCTTCCGATCTAACACCTTCATC | SEQ ID |
| | ATCCGCTGC | NO: 91 |
| Adp_fp_Mela8 | ACACGACGCTCTTCCGATCTTCAAAAGGAAGT | SEQ ID |
| | ATCTTGGCCTCC | NO: 92 |
| Adp_fp_Mela9 | ACACGACGCTCTTCCGATCTCAGTGCGCTTTT | SEQ ID |
| | CCCAACA | NO: 93 |
| Adp_fp_Mela10 | ACACGACGCTCTTCCGATCTTGTTGGACATAC | SEQ ID |
| | TGGATACAGCT | NO: 94 |
| Adp_fp_Mela11 | ACACGACGCTCTTCCGATCTGTCAAGGCACTC | SEQ ID |
| | TTGCCTAC | NO: 95 |
| Adp_fp_Mela12 | ACACGACGCTCTTCCGATCTTCTTGGATATTC | SEQ ID |
| | TCGACACAGCA | NO: 96 |
| Adp_fp_Mela13 | ACACGACGCTCTTCCGATCTGCAATTTCTACA | SEQ ID |
| | CGAGATCCTCTC | NO: 97 |
| Adp_fp_Mela14 | ACACGACGCTCTTCCGATCTTTGGAGTATTTC | SEQ ID |
| | ATGAAACAAATGAATGAT | NO: 98 |
| Adp_fp_Mela15 | ACACGACGCTCTTCCGATCTTGGGACCCACTC | SEQ ID |
| | CATCGAGAT | NO: 99 |
| BDA_fp_Mela1 | CCTTGAGGCCTTTCTTACCCA | SEQ ID |
| | | NO: 100 |
| BDA_fp_Mela2 | AGCTGCAGGTTCTGCATGA | SEQ ID |
| | | NO: 101 |
| BDA_fp_Mela3 | CGCTGACCCCAAAGTCACA | SEQ ID |
| | | NO: 102 |
| BDA_fp_Mela4 | TTCGCCGACCTTGGCT | SEQ ID |
| | | NO: 103 |
| BDA_fp_Mela5 | GCTGCAGGTCCTGCACG | SEQ ID |
| | | NO: 104 |
| BDA_fp_Mela6 | CATCCTCGTGAACTCTAGAGG | SEQ ID |
| | | NO: 105 |
| BDA_fp_Mela7 | AACACCTTCATCATCCGCTGC | SEQ ID |
| | | NO: 106 |
| BDA_fp_Mela8 | TCAAAAGGAAGTATCTTGGCCTCC | SEQ ID |
| | | NO: 107 |
| BDA_fp_Mela9 | CAGTGCGCTTTTCCCAACA | SEQ ID |
| | | NO: 108 |
| BDAfpMela10 | TGTTGGACATACTGGATACAGCT | SEQ ID |
| | | NO: 109 |
| BDA_fp_Mela11 | GTCAAGGCACTCTTGCCTAC | SEQ ID |
| | | NO: 110 |
| BDA_fp_Mela12 | TCTTGGATATTCTC GACACAGGA | SEQ ID |
| | | NO: 111 |
| BDA_fp_Mela13 | GCAATTTCTACACGAGATCCTCTC | SEQ ID |
| | | NO: 112 |
| BDA_fp_Mela14 | TTGGAGTATTTCATGAAACAAATGAATGAT | SEQ ID |
| | | NO: 113 |
| BDA_fp_Mela15 | TGGGACCCACTCCATCGAGAT | SEQ ID |
| | | NO: 114 |
| BDA_BlockerMela1 | TCTTACCCAGAAGCAGAAGGTGGGA/iSpC3// | SEQ ID |
| | iSpC3/AA | NO: 115 |
| BDA_BlockerMela2 | CTGCATGAGTGCAACTCTCCGTACA/iSpC3// | SEQ ID |
| | iSpC3/GC | NO: 116 |
| BDA_Blocker_Mela3 | AAAGTCACAGAGCTTGATCTCCCCAC/iSpC3// | SEQ ID |
| | iSpC3/GC | NO: 117 |
| BDA_Blocker_Mela4 | TTGGCTTTCTGGGTGAGAAAGGCTT/iSpC3// | SEQ ID |
| | iSpC3/AC | NO: 118 |
| BDA_Blocker_Mela5 | CTGCACGAATGCAACTCGCCGTA/iSpC3// | SEQ ID |
| | iSpC3/TA | NO: 119 |
| BDA_Blocker_Mela6 | TCTAGAGGGGAGATCAAGCTGTGTGA/ | SEQ ID |
| | iSpC3//iSpC3/AA | NO: 120 |
| BDA_Blocker_Mela7 | CGCTGCCTGCAGTGGACCACT/iSpC3// | SEQ ID |
| | iSpC3/CC | NO: 121 |
| BDA_Blocker_Mela8 | GCCTCCAGTTTTTTATATATTCTCCTACATGA | SEQ ID |
| | GG/iSpC3//iSpC3/AA | NO: 122 |
| BDA_Blocker_Mela9 | CCCAACACCACCTGCTCCAACC/iSpC3// | SEQ ID |
| | iSpC3/CT | NO: 123 |
| BDA_Blocker_Mela10 | GATACAGCTGGAGAAGAAGAGTACAGTG/ | SEQ ID |
| | iSpC3//iSpC3/AC | NO: 124 |
| BDA_Blocker_Mela11 | TGCCTACGCCACCAGCTCCA/iSpC3// | SEQ ID |
| | iSpC3/TT | NO: 125 |
| BDA_Blocker_Mela12 | CACAGCAGGTCAAGAGGAGTACAGTG/ | SEQ ID |
| | iSpC3//iSpC3/AC | NO: 126 |
| BDA_Blocker_Mela13 | ATCCTCTCTCTGAAATCACTGAGCAGG/ | SEQ ID |
| | iSpC3//iSpC3/AC | NO: 127 |
| BDA_Blocker_Mela14 | CAAATGAATGATGCACATCATGGTGGC/ | SEQ ID |
| | iSpC3//iSpC3/GT | NO: 128 |
| BDA_Blocker_Mela15 | CCATCGAGATTTCACTGTAGCTAGACCAAAA/ | SEQ ID |
| | iSpC3/iSpC3/AA | NO: 129 |
| Reverse Blocker | CTGAGATCAGCCAAAGTCAGTTATTTTTTCTG/ | SEQ ID |
| Mela 13 | iSpC3//iSpC3/AC | NO: 130 |

TABLE 6
Melanoma QBDA Panel validation using gBlock Spike-in

| | | | | QBDA | | |
|---|---|---|---|---|---|---|
| | | | | Obs. | QBDA | Expected |
| | gBlock spike-in | | | Var | Calc. | gBlcok |
| Target | Gene | mutation | COSMIC ID | Mol | VAF | VRF |
| 1 | MAP2K1 | K57E | COSM5369532 | 106 | 0.9% | 0.9% |
| 2 | | C121S | COSM555601 | 124 | 1.1% | 0.9% |
| 3 | | E203K | COSM232755 | 202 | 1.3% | 1.0% |
| 4 | MAP2K2 | F57V | COSM3534171 | 126 | 1.6% | 0.9% |
| 5 | | C125S | COSM5855815 | 182 | 1.4% | 1.7% |
| 6 | | E207K | COSM5574290 | 152 | 2.0% | 1.3% |
| 7 | AKT1 | Q79K | COSM159008 | 174 | 1.3% | 1.0% |
| 8 | AKT3 | E17K | COSM224779 | 173 | 1.8% | 1.2% |
| 9 | NRAS | G12S | COSM563 | 285 | 1.6% | 0.9% |
| 10 | | Q61K | COSM580 | 167 | 1.4% | 0.9% |
| 11 | KRAS | G12S | COSM517 | 145 | 1.9% | 1.6% |
| 12 | | Q61K | COSM87298 | 101 | 1.1% | 1.3% |
| 13 | PIK3CA | E542K | COSM760 | 148 | 2.1% | 0.8% |
| 14 | | H1047Y | COSM774 | 99 | 1.0% | 1.2% |
| 15 | BRAF | V600E | COSM476 | 197 | 1.8% | 0.6% |

TABLE 7
QBDA Melanoma panel test using clinical samples (Fresh Frozen and FFPE)

| | Mutation in | Mutation | | | | Deep |
|---|---|---|---|---|---|---|
| | Enrichment | in | | Obs. Var | | sequencing |
| Sample ID | region | Genome | Amino Acid Change | Mol # | Calc. VAF | VRF |
| FF1 | Tar15, | Chr7: | BRAF(c.1799T > A; | 6239 | 87.5% | 55.8% |
| | 4A > T, | 140753336 | p.V600E) | | | |
| | | A > T | | | | |
| FF2 | No Variants >0.1% | | | | | |
| FF3 | No Variants >0.1% | | | | | |
| FF4 | Tar10, | Chr1: | NRAS(c.182A > T; | 2217 | 29.1% | 27.2% |
| | 5A > T | 114713908 | p.Q61L) | | | |
| | | A > T | | | | |
| FFPE1 | Tar10, | Chr1: | NRAS(c.182A > G; | 308 | 4.04% | 8.9% |
| | 5A > G | 114713908 | p.Q61R) | | | |
| | | A > G | | | | |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Wu, L. R., Chen, S. X., Wu, Y., Patel, A. A., & Zhang, D. Y. (2017). Multiplexed enrichment of rare DNA variants via sequence-selective and temperature-robust amplification. Nature biomedical engineering, 1(9), 714.
2. U.S. patent application Ser. No. 15/355,235, published as US-2017-0067090 on Mar. 9, 2017.
3. U.S. patent application Ser. No. 16/971,411, published as US-2021-0024989 on Jan. 28, 2021.
4. International Patent Application No. PCT/US2020/012089, published as WO 2020/142631 on Jul. 9, 2020.

What is claimed is:

1. A method for labeling and amplifying each strand of between 1 and 10,000 target genomic regions with an oligonucleotide barcode sequence by polymerase chain reaction (PCR), the method comprising:
(a) introducing into a DNA sample comprising the between 1 and 10,000 target genomic regions, for each target genomic region:
(i) a first oligonucleotide, comprising from 5′ to 3′ end, (A) a first region, (B) a second region with a length between 0 and 50 nucleotides, and (C) a third region targeting a first specific genomic region; and
(ii) a second oligonucleotide, comprising from 5′ to 3′ end, (A) a fourth region, (B) a fifth region with a length between 0 and 50 nucleotides, (C) a sixth region comprising a unique molecular identifier (UMI) comprising at least four degenerate nucleotides, and (D) a seventh region targeting a second specific genomic region;
(b) performing at least two cycles of PCR amplification to generate a first PCR amplification product;
(c) introducing into the first PCR amplification product:
(i) a third oligonucleotide comprising the first region; and
(ii) a fourth oligonucleotide, comprising the fourth region;
(d) performing at least two cycles of PCR amplification, to generate a second PCR amplification product;
(e) introducing to the second PCR amplification product:
(i) a fifth oligonucleotide (Blocker Displacement Amplification (BDA) forward primer) for each target genomic region, wherein the BDA forward primer comprises an eighth region targeting a specific genomic region, wherein the genomic region targeted by the eighth region is between 1 and 20 nucleotides closer to the seventh region compared to the genomic region targeted by the third region,
(ii) a sixth oligonucleotide (BDA blocker) for each target genomic region, wherein 4 or more nucleotides at the 3′ end of the BDA forward primer sequence are also present at or near the 5′ end of the BDA blocker sequence; and
wherein the BDA blocker contains a 3′ sequence or modification that prevents extension by a DNA polymerase, and wherein the concentration of the BDA blocker is at least 2 times that of the BDA forward primer, and
(iii) a seventh oligonucleotide, comprising the fourth region; and
(f) performing at least two cycles of PCR amplification to generate a third PCR amplification product.

2. The method of claim 1, wherein the first region in the first oligonucleotide in step (a) and the fourth region in the second oligonucleotide in step (a) generate binding sites for universal amplification performed in step (c).

3. The method of claim 1 or 2, wherein the fourth region in the second oligonucleotide comprises at least part of the next-generation sequencing (NGS) adapter sequence.

4. The method of any one of claims 1-3, wherein the melting temperatures of the first and the fourth regions are between 0.01° C. and 10° C. higher than the melting temperatures of the third and the seventh regions.

5. The method of any one of claims 1-4, wherein the degenerate nucleotides in the sixth region each independently are one of A, T, or C.

6. The method of any one of claims 1-5, wherein the first PCR amplification product from step (d) is purified prior to step (e) using a method selected from the group consisting of SPRI purification, column purification, and enzymatic digestion.

7. The method of any one of claims 1-6, wherein step (a) further comprises introducing into the DNA sample a blocker oligonucleotide that comprises from 5′ to 3′ end, sequence that targets a pseudogene or other undesired genomic region and 3′ sequence or modification that prevents extension by DNA polymerase.

8. The method of any one of claims 1-7, further comprising:
(g) introducing to the PCR amplification product obtained in step (f), (i) an eighth oligonucleotide, comprising from 5′ to 3′ end, a ninth region and an eighth region, wherein the ninth region comprises at least part of the next-generation sequencing (NGS) adapter sequence, and optionally (ii) a ninth oligonucleotide, comprising the fourth region; and
(h) performing at least one cycle of PCR amplification to obtain a third PCR amplification product.

9. The method of any one of claims 1-8, further comprising:
(i) adding NGS adapter sequences to the PCR amplification product obtained in step (f) by ligation reaction.

10. The method of claim 8 or 9, further comprising adding NGS indices by PCR and purifying the third PCR amplification product.

11. The method of claim 10, wherein the purifying comprises SPRI purification, column purification, or enzymatic digestion.

12. The method of claim 10 or 11, further comprising performing high-throughput DNA sequencing.

13. The method of claim 12, wherein the high-throughput DNA sequencing is next-generation sequencing.

14. The method of any one of claims 1-13, wherein an annealing temperature used in step (d) is between 0.01° C. and 10° C. higher than an annealing temperature used in step (b).

15. The method of any one of claims 1-14, wherein at least one of the between 1 and 10,000 target genomic regions is selected from the group consisting of is selected from the group consisting of AKT1, ALK, APC, AR, ATM, BRAF, CCND1, CDK4, CDKN2A, CHEK2, CTNNB1, DDR2, EGFR, ERBB2, ERBB3, ERBB4, ESR1, EZH2, FBXW7, FGFR1, FGFR2, FGFR3, FGFR4, FLT3, FOXL2, GNA11, GNAQ, GNAS, HRAS, IDHL JAK1, JAK2, JAK3, KIT, KRAS, MAP2K1, MAP2K2, MET, MLH1, MPL, MTOR, MYC, MYCN, MYD88, NPM1, NRAS, PDGFRA, PIK3CA, PTEN, PTPN11, RAF1, RB1, RET, ROS1, SF3B1, SMAD4, SMARCB1, SMO, STK11, and TP53.

16. A method for quantitating the variant allele frequency (VAF) of variant sequences in between 1 and 10,000 target genomic regions, the method comprising:
(a) designing a panel of oligonucleotides and blockers for the target genomic regions;
(b) labeling and amplifying each strand of the targeted genomic regions according to the method of any one of claims 1-15; and
(c) determining the VAF of variant sequences based on high-throughput sequencing data and the input amount of DNA sample.

17. The method of claim 16, wherein step (a) comprises:
(i) designing a primer set for each selected genomic region; each primer set containing the first, the second, the fifth, the sixth, and the eighth oligonucleotide are as described in claims 1-15;
(ii) designing the third and the fourth oligonucleotide to be used for universal amplification of all selected genomic regions; and
(iii) checking the specificity of the primer set in whole genome to ensure that the primers are not prone to nonspecific amplification of non-target regions.

18. The method of claim 16 or 17, wherein step (c) comprises:
(i) aligning NGS reads to the targeted amplicon regions, and grouping the NGS reads into region-specific subgroups by the regions to which they are aligned;
(ii) at each locus, dividing the NGS reads by the UMI sequence, where all NGS reads carrying the UMI sequence are grouped as one UMI family;
(iii) removing UMI families that are likely results of PCR or NGS errors;
(iv) determine the genotype for each remaining UMI family;
(v) counting the unique UMI number N (the total count of different UMI sequences at one locus) for each variant sequence at each targeted genetic region, which indicates the number of original strands; and
(vi) calculating VAF for a variant sequence as VAF=Nᵥₐᵣ/(Nᵢₙₚᵤₜ*Yield), where Nᵥₐᵣ is unique UMI number for the variant sequence, Nᵢₙₚᵤₜ is strand number of DNA input for QBDA, and Yield is the overall conversion yield for QBDA reaction.

19. The method of claim 18, wherein UMI families are considered to be likely results of PCR or NGS errors if the UMI sequence does not meet the UMI degenerate base design pattern or the UMI family has a UMI family size <Fₘᵢₙ, wherein Fₘᵢₙ is between 2 and 20.

20. The method of claim 18 or 19, wherein step (iv) comprises determining the genotype supported by at least 70% of the reads in the same UMI family.

21. The method of claim 18 or 19, wherein step (iv) comprises determining the genotype as wild type (WT), if WT reads is supported by more than P_{WT} reads in the UMI family, wherein P_{WT} is 0.01%-50%.

22. The method of any one of claims 18-21, wherein step (v) further comprises removing UMI sequences that differs by only 1 or 2 bases from another UMI with a larger family size.

23. A method for labeling and amplifying each strand of at least one target genomic region with an oligonucleotide barcode sequence by polymerase chain reaction (PCR), the method comprising:
(a) adding a unique molecular identifier (UMI) to the at least one target genomic region;
(b) amplifying the at least one target genomic region from step (a) using a universal forward primer and a universal reverse primer, to generate a first PCR amplification product, wherein the at least one target genomic region comprises the UMI; and
(c) amplifying the first PCR amplification product using a Blocker Displacement Amplification (BDA) forward primer, a BDA blocker, and a universal reverse primer to generate a second PCR amplification product.

24. The method of claim 23, wherein step (a) comprises at least 2 cycles of PCR amplification.

25. The method of claim 23 or 24, wherein step (b) comprises at least 2 cycles of PCR amplification.

26. The method of any one of claims 23-25, wherein step (c) comprises at least 2 cycles of PCR.

27. The method of any one of claims 23-26, wherein the BDA blocker comprises a 3′ sequence or modification that prevents extension by a DNA polymerase.

28. The method of any one of claims 23-27, wherein 4 or more nucleotides at the 3′ end of the BDA forward primer sequence are also present at or near the 5′ end of the BDA blocker sequence.

29. The method of any one of claims 23-28, wherein the concentration of the BDA blocker is at least 2 times that of the BDA forward primer.

30. A method for labeling and amplifying each strand of at least one target genomic region with an oligonucleotide barcode sequence by polymerase chain reaction (PCR), the method comprising:
(a) introducing into a DNA sample comprising the at least one target genomic region:
(i) a first oligonucleotide, comprising a third region targeting a first specific genomic region; and
(ii) a second oligonucleotide, comprising a region comprising a unique molecular identifier (UMI) comprising at least four degenerate nucleotides, and a region targeting a second specific genomic region;
(b) performing at least two cycles of PCR amplification to generate a first PCR amplification product;
(c) introducing to the first PCR amplification product:
(i) a universal forward primer; and
(ii) a universal reverse primer;
(d) performing at least two cycles of PCR amplification, to generate a second PCR amplification product;
(e) introducing to the second PCR amplification product:
(i) a Blocker Displacement Amplification (BDA) forward primer targeting the specific genomic region, wherein the genomic region targeted by the eighth region is between 1 and 20 nucleotides closer to the seventh region compared to the genomic region targeted by the third region,
(ii) a BDA blocker for the target genomic region, wherein 4 or more nucleotides at the 3′ end of the BDA forward primer sequence are also present at or near the 5′ end of the BDA blocker sequence; and wherein the BDA blocker contains a 3′ sequence or modification that prevents extension by a DNA polymerase, and
(iii) a universal reverse primer; and
(f) performing at least two cycles of PCR amplification to obtain a third PCR amplification product.

31. The method of claim 30, wherein the second PCR amplification product is purified between step (d) and step (e).

32. The method of claim 30 or 31, wherein the concentration of the BDA blocker is at least 2 times that of the BDA forward primer.

33. A method for labeling and amplifying each strand of between 1 and 10,000 target genomic regions with an oligonucleotide barcode sequence by polymerase chain reaction (PCR), the method comprising:
(a) introducing into a DNA sample comprising the between 1 and 10,000 target genomic regions, for each target genomic region:
(i) a first oligonucleotide, comprising from 5′ to 3′ end, (A) a first region, (B) a second region with a length between 0 and 50 nucleotides, and (C) a third region targeting a first specific genomic region; and
(ii) a second oligonucleotide, comprising from 5′ to 3′ end, (A) a fourth region, (B) a fifth region with a length between 0 and 50 nucleotides, (C) a sixth region comprising a unique molecular identifier (UMI) comprising at least four degenerate nucleotides, and (D) a seventh region targeting a second specific genomic region;
(b) performing at least two cycles of PCR amplification to generate a first PCR amplification product;
(c) introducing into the first PCR amplification product:
(i) a third oligonucleotide comprising the first region; and
(ii) a fourth oligonucleotide, comprising the fourth region;
(d) performing at least two cycles of PCR amplification, wherein the annealing temperature is between 0.01° C. and 10° C. higher than an annealing temperature used in step (b) to generate a second PCR amplification product;
(e) purifying the second PCR amplification product to remove single-stranded primers;
(f) introducing to the second PCR amplification product purified in step (e):
(i) a fifth oligonucleotide (Blocker Displacement Amplification (BDA) forward primer) for each target genomic region, wherein the BDA forward primer comprises an eighth region targeting a specific genomic region, wherein the genomic region targeted by the eighth region is between 1 and 20 nucleotides closer to the seventh region compared to the genomic region targeted by the third region,
(ii) a sixth oligonucleotide (BDA blocker) for each target genomic region, wherein 4 or more nucleotides at the 3′ end of the BDA forward primer sequence are also present at or near the 5′ end of the BDA blocker sequence; and wherein the BDA blocker contains a 3′ sequence or modification that prevents extension by a DNA polymerase, and wherein the concentration of the BDA blocker is at least 2 times that of the BDA forward primer, and
(iii) a seventh oligonucleotide, comprising the fourth region; and
(g) performing at least two cycles of PCR amplification to generate a third PCR amplification product.

34. A kit for labeling and amplifying each strand of at least 1 target genomic region with an oligonucleotide barcode sequence by polymerase chain reaction, the kit comprising:
(a) a DNA polymerase;
(b) dNTPs;
(c) at least one Blocker Displacement Amplification (BDA) forward primer;
(d) at least one BDA blocker;
(e) at least one universal forward primer;
(f) at least one universal reverse primer; and
(g) at least one oligonucleotide comprising a Unique Molecular Identifier.

35. The kit of claim 34, wherein the kit further comprises (h) a DNA polymerase buffer.

36. The kit of claim 34 or 35, wherein the kit further comprises (i) nuclease-free water.
